(19) United States
(12) Patent Application Publication
(10) Pub. No.: US 2024/0299483 A1
(43) Pub. Date: Sep. 12, 2024

(54) TARGETS AND BIOMARKERS FOR TUMOR MICROENVIRONMENTS

(71) Applicant: The Trustees of Indiana University, Bloomington, IN (US)

(72) Inventors: Cong Yan, Carmel, IN (US); Hong DU, Carmel, IN (US)

(21) Appl. No.: 18/599,502

(22) Filed: Mar. 8, 2024

Related U.S. Application Data
(60) Provisional application No. 63/451,067, filed on Mar. 9, 2023.

(51) Int. Cl. A61K 38/00; A61K 45/06; A61P 35/00

(52) U.S. Cl. CPC A61K 38/005; A61K 45/06; A61P 35/00

(57) ABSTRACT

The present disclosure relates to methods and compositions that inhibit a tumor microenvironment and methods of co-administering such compositions to inhibit the tumor microenvironment and treat cancer in a subject.

### CROSS-REFERENCE TO RELATED APPLICATIONS

This application claims benefit to U.S. Provisional Application No. 63/451,067, filed Mar. 9, 2023. The contents of which are incorporated herein by reference in their entirety.

### GOVERNMENT LICENSED RIGHTS

This invention was made with government support under CA225108 awarded by the National Institutes of Health. The Government has certain rights in the invention.

### BACKGROUND/FIELD

The present disclosure relates to methods and compositions for inhibiting tumor microenvironments. More particularly, the present disclosure relates to methods and compositions for inhibiting tumor microenvironments and treating cancer in a subject.

### SUMMARY

The present disclosure takes advantage of the recently discovered molecular mechanisms that impart various cancer-advantageous properties to tumor microenvironments. Without being limited by theory, it is believed that tumor microenvironments are capable of forming an ecosystem that 1) encourages the growth and expansion of cancer cells and 2) inhibits a natural tumor suppressor response by a subject's immune system. As a result, the disclosure provides methods and compositions to inhibit the tumor microenvironment by reducing the growth and expansion of cancer cells, and reducing or eliminating the suppression of a subject's immune response to cancer cells. The methods and compositions may be useful to treat cancer in a subject.

Additionally, the recently discovered molecular mechanisms have identified new biomarkers for detecting a cancer. In particular, the cancer may be non-small cell lung cancer (NSCLC).

The present disclosure relates to a method of inhibiting a tumor microenvironment in a subject comprising administering a therapeutically effective amount of a composition comprising a lysosomal acid lipase (LAL) replacement therapy, a pyruvate dehydrogenase (PDH) inhibitor, a programmed death-ligand 1 (PD-L1) inhibitor, or a combination thereof.

In another aspect, a method of treating a cancer in a subject is disclosed comprising 1) inhibiting a tumor microenvironment comprising a) administering a therapeutically effective amount of a composition including an LAL replacement therapy, a PDH inhibitor, a PD-L1 inhibitor, or a combination thereof; and 2) administering an anti-tumor treatment.

The tumor microenvironment may include myeloid derived suppressor cells (MDSCs) characterized by the expression of one or more CD11b+, CD13+, CD14+, CD15+, CD33+, and/or HLA-DR-. In some embodiments, the myeloid derived suppressor cells (MDSCs) have a reduced protein expression level of lysosomal acid lipase (LAL) compared to a myeloid cell not in a tumor microenvironment. In some embodiments, the MDSCs are characterized by an inhibition of LAL protein expression.

### BRIEF DESCRIPTION OF THE FIGURES

FIG. 1A measures the number of cells positive for lymphocyte antigen 6 member G and lymphocyte antigen 6 C1 (identified as Ly6G⁺Ly6C⁺ cells), isolated from the blood of wild type (lysosomal acid lipase (LAL) expressed, denoted as Lal^{+/+}) Friend Virus B/NIH (FVB/N) mice and LAL knockout (denoted as Lal^{-/-}) FVB/N mice.

FIG. 1B measures the number of cells positive for lymphocyte antigen member G and lymphocyte antigen 6 C1 (identified as Ly6G⁺Ly6C⁺ cells), isolated from the bone marrow of wild type (lysosomal acid lipase (LAL) expressed, denoted as Lal^{+/+}) Friend Virus B/NIH (FVB/N) mice and LAL knockout (denoted as Lal^{-/-}) FVB/N mice.

FIG. 1C measures the number of polymorphonuclear—MDSCs, cells positive for integrin alpha M and Ly6G (identified as CD11b⁺Ly6G⁺ cells), from flank tissue isolated from Lal^{+/+} mice and Lal^{-/-} mice. Percentages of CD11b⁺Ly6G⁺ cells from these mice were compared to CD11b⁺Ly6G⁺ cells from tissues of Lal^{+/+} mice and Lal^{-/-} mice injected with B16 melanoma cells in the flank sites. Fourteen days after injection, tumor tissues from B16 melanoma-injected Lal^{+/+} mice and Lal^{-/-} mice were harvested. Percentages of all CD11b⁺Ly6G⁺ cells were analyzed by flow cytometry.

FIG. 2A is a western blot analysis of the protein expression of metabolic enzymes in Lal^{-/-} Ly6G⁺ cells versus Lal^{+/+} Ly6G⁺ cells. Enzymes specifically tested were HKI(hexokinase 1), HK2 (hexokinase 2), HK3 (hexokinase 3) and PDH (pyruvate dehydrogenase) in the glycolytic pathway, G6PD (glucose-6-phosphate dehydrogenase) in the pentose phosphate pathway, and LDH (lactate dehydrogenase). Protein expression of all enzymes were increased in Lal^{-/-} Ly6G⁺ cells.

FIG. 2B shows levels of glucose (the entry substrate of glycolysis), pyruvate (the end product of glycolysis) and α-ketoglutarate (a key molecule in the citric acid cycle) in bone marrow Ly6G⁺ cells from Lal^{-/-} and Lal^{+/+} mice. Glucose, pyruvate and α-ketoglutarate were measured through glucose assay kit; pyruvate assay kit; and α-ketoglutarate assay kit from Sigma Aldrich.

FIG. 3A shows percentage of CD11b⁺Ly6G⁺ cells measured from bone marrow of Lal^{-/-} and Lal^{+/+} mice. Mice received 5 mg/kg CPI-613 (PDH inhibitor) on days 0, 2, 4, and 7. On day 8, the CD11b⁺Ly6G⁺ population in the bone marrow was determined by flow cytometry. DMSO (dimethyl sulfoxide) was used as a control.

FIG. 3B shows ROS production in Lal^{-/-} and Lal^{+/+} Ly6G⁺ cells after CPI-613 treatment.

FIG. 3C shows T-cell proliferation after cocultured with CPI-613-pretreated Lal^{+/+} or Lal^{-/-} Ly6G⁺ cells in vivo. Cluster of Differentiation 4 Protein-positive white blood cells (CD4⁺ T cells) were labeled with carboxyfluorescein succinimidyl ester (CSFE) and cocultured with CPI-613-pretreated Lal^{+/+} or Lal^{-/-} Ly6G⁺ cells which were then used to measure T-cell proliferation through T-cell proliferation assay. Peaks represent cell division cycles. DMSO was used as a negative control. A representative CFSE dilution by flow cytometry is on the left. Statistical analyses of percent of divided CD4⁺ T cells is on the right.

FIG. 3D shows proliferation of B16 melanoma and Lewis lung carcinoma (LLC) cells after cocultured with CPI-613-pretreated Lal^{+/+} or Lal^{-/-} Ly6G⁺ cells in vitro for 72 hours. Cell number in 10³ is seen, with B16 melanoma cells measured on the left and LLC cells measured on the right.

FIG. 3E shows size of tumor in mm³ of B16 melanoma cells after coinjection with CPI-613-pretreated Lal^{+/+} or Lal^{-/-} Ly6G⁺ cells in vivo. Tumor size was measured at 10 days.

FIG. 3F is a western blot analysis of the protein expression of PDH in Lal^{-/-} Ly6G⁺ cells in vitro after small interfering RNA (siRNA) knockdown of PDH. Actin was measured as a control.

FIG. 3G shows measure of ROS production through flow cytometry of Lal^{+/+} or Lal^{-/-} Ly6G⁺ cells 24 hours after transfection of PDH siRNA.

FIG. 3H shows proliferation of B16 melanoma (left) and LLC (right) cells after cocultured with PDH siRNA-transfected Lal^{+/+} versus Lal^{-/-} Ly6G⁺ cells in vitro.

FIG. 3I is a western blot analysis of the protein expression of programmed death ligand-1 (PD-L1) in Lal^{+/+} or Lal^{-/-} Ly6G⁺ cells. Actin was measured as a control.

FIG. 3J measures PD-L1⁺ cells of Ly6G⁺ CD11c⁺ cells isolated from Lal^{+/+} and Lal^{-/-} mice. Mice received 5 mg/kg CPI-613 on days 0, 2, 4, and 7. On day 8, the percentage of PD-L1⁺ cells in Ly6G⁺ CD11c⁺ cells was measured by flow cytometry analysis.

FIG. 4A shows a representative gating strategy of LAL⁺ cells in the following myeloid cell populations: CD11b⁺ HLA-DR⁻; CD13⁺; CD14⁺; CD15⁺; CD33⁺; CD11b⁺ CD13⁺HLA-DR⁻; CD11b⁺ CD14⁺ HLA-DR⁻; CD11b⁺ CD15⁺HLA-DR⁻; and CD11b⁺ CD33⁺ HLA-DR⁻ cells.

FIG. 4B is a measurement of mean fluorescent intensity (MFI) of whole blood and myeloid cell subsets of patients with non-small cell lung cancer (NSCLC) patients (right) and healthy subjects (left).

FIG. 4C shows statistical analyses of percentage of LAL⁺ cells in whole blood and myeloid cell subsets of patients with non-small cell lung cancer (NSCLC) patients (right) and healthy subjects (left).

FIG. 5A shows a representative gating strategy of CD13⁺, CD14⁺, CD15⁺, and CD33⁺ cells in the leucocytes from NSCLC patients versus healthy subjects.

FIG. 5B shows statistical analyses of percentages of CD11b⁺ HLA-DR⁻, CD13⁺, CD14⁺, CD15⁺, and CD33⁺ cells in the leucocytes from NSCLC patients versus healthy subjects. Cells were analyzed through flow cytometry.

FIG. 5C shows statistical analysis of percentages of CD13⁺, CD14⁺, CD15⁺, and CD33⁺ cells in CD11b⁺ HLA-DR⁻ cells from NSCLC patients versus healthy subjects. Cells were analyzed through flow cytometry.

FIG. 5D shows statistical analysis of percentages of CD11b⁺ HLA-DR⁻, CD11b⁺ CD13⁺ HLA-DR⁻, CD11b⁺ CD14⁺ HLA-DR⁻, and CD11b⁺ CD15⁺ HLA-DR⁻ cells. Human leucocytes from healthy individuals were incubated with 10 μM Lalistat2, an LAL inhibitor (L) or DMSO (S) for 24 hours; control group (C) were not treated. After 24 hours, cells were analyzed by flow cytometry.

FIG. 6A shows a representative gating strategy of CD13⁺ and CD14⁺ cells in the blood of Lal^{+/+} and Lal^{-/-} mice.

FIG. 6B shows statistics of percentages of CD13⁺ and CD14⁺ cells in the blood of Lal^{+/+} and Lal^{-/-} mice.

FIG. 6C shows a representative gating strategy of CD13⁺ and CD14⁺ cells in the bone marrow of Lal^{+/+} and Lal^{-/-} mice.

FIG. 6D shows statistics of percentages of CD13⁺ and CD14⁺ cells in the bone marrow of Lal^{+/+} and Lal^{-/-} mice.

FIG. 7A shows a representative gating strategy of PDH⁺, G6PD⁺, LDH⁺, and GLUD⁺ cells in whole blood cells of NSCLC patients versus healthy subjects.

FIG. 7B shows MFI (top row) and percentages (bottom row) of PDH⁺, G6PD⁺, LDH⁺, and GLUD⁺ cell in the leucocytes of NSCLC patients versus healthy subjects.

FIG. 7C shows a representative gating strategy of PDH⁺, G6PD⁺, LDH⁺, and GLUD⁺ cells in blood CD11b⁺ HLA-DR⁻ cells of NSCLC patients versus healthy subjects.

FIG. 7D shows MFI (top row) and percentages (bottom row) of PDH⁺, G6PD⁺, LDH⁺, and GLUD⁺ cell blood CD11b⁺ HLA-DR⁻ cells of NSCLC patients (right) versus healthy subjects (left).

FIG. 7E shows MFI and percentages of PDH⁺, G6PD⁺, LDH⁺, and GLUD⁺ cell blood CD11b⁺ CD13⁺HLA-DR⁻ cells of NSCLC patients (right) versus healthy subjects (left).

FIG. 8A shows statistical analysis of percentages of PD-L1⁺ cells in leucocytes of patients with NSCLC before versus after anti-PD-L1 treatment with PD-L1 checkpoint inhibitor.

FIG. 8B shows statistical analysis of percentages of CD11b⁺ HLA-DR⁻, CD13⁺, CD14⁺, CD15⁺, and CD33⁺ cells in the leucocytes of patients with NSCLC (left) before versus after anti-PD-L1 treatment with PD-L1 checkpoint inhibitor (right).

FIG. 8C shows statistical analysis of percentages of CD13⁺, CD14⁺, CD15⁺, and CD33⁺ cells in CD11b⁺ HLA-DR⁻ cells of patients with NSCLC before versus after anti-PD-L1 treatment with PD-L1 checkpoint inhibitor.

FIG. 8D shows MFI of PDH⁺, G6PD⁺, LDH⁺, and GLUD⁺ cells in the leucocytes of patients with NSCLC before versus after anti-PD-L1 treatment with PD-L1 checkpoint inhibitor.

FIG. 8E shows MFI of PDH⁺, G6PD⁺, LDH⁺, and GLUD⁺ cells in blood CD11b+ HLA-DR⁻ cells of patients with NSCLC before versus after anti-PD-L1 treatment with PD-L1 checkpoint inhibitor.

FIG. 8F shows MFI of PDH⁺, G6PD⁺, LDH⁺, and GLUD⁺ cells in blood CD11b⁺ CD13⁺ HLA-DR⁻ cells of patients with NSCLC before versus after anti-PD-L1 treatment with PD-L1 checkpoint inhibitor.

FIG. 9 shows the chemical structure of CPI-613.

FIG. 10 shows the chemical structure of pexidartinib.

### DETAILED DESCRIPTION

The present disclosure may be further understood by reference to the following detailed description.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “pharmaceutical composition” refers to a mixture of a compound described herein with other chemical components, such as carriers, stabilizers, diluents, dispersing agents, suspending agents, thickening agents, and/or excipients.

As used herein, the term “tumor microenvironment” or “TME” means a group of cells including myeloid derived suppressor cells (MDSCs) capable of forming a pro-tumor environment and/or support the growth and expansion of a tumor including increased production of reactive oxygen species (ROS). Additionally, the TME comprising MDSCs are capable of suppressing a subject's natural immune response to a tumor or cancer cell. The TME may be present before a tumor forms or after a tumor forms.

As used herein, the term “myeloid derived suppressor cells” or “MDSCs” are characterized by the expression of CD11b+, CD13+, CD14+, CD15+, CD33+, HLA-DR−, or a combination thereof. Further, the MDSCs may be identified by a reduced or inhibited expression of lysosomal acid lipase (LAL).

As used herein, a “tumor” means an abnormal mass of tissue that forms when cells grow and divide more than they should or do not die when they should.

As used herein, “inhibiting a tumor microenvironment” means reducing or inhibiting the growth of a tumor, reducing the production of ROS products, and/or reducing the immunosuppressive response of the tumor microenvironment.

As used herein, “therapeutically effective amount” means the amount which, when administered to a subject or patient for treating a disease, is sufficient to effect such treatment for the disease, including to ameliorate at least one symptom of the disease.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; a reduction in the production of reactive oxygen species, inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

A method of inhibiting a tumor microenvironment in a subject is disclosed comprising administering a therapeutically effective amount of a composition including a lysosomal acid lipase (LAL) replacement therapy, a pyruvate dehydrogenase (PDH) inhibitor, a programmed death-ligand 1(PD-L1) inhibitor, or a combination thereof. In some embodiments, the method further comprises administering a colony-stimulating factor 1 receptor (CSF1R).

In some embodiments, a method of treating a cancer in a subject comprises inhibiting a tumor microenvironment comprising administering a therapeutically effective amount of a composition including a lysosomal acid lipase (LAL) replacement therapy, a pyruvate dehydrogenase (PDH) inhibitor, a programmed death-ligand 1(PD-L1) inhibitor, or a combination thereof; and administering an anti-tumor treatment. In some embodiments, a method of detecting a tumor microenvironment comprises analyzing the presence of myeloid derived suppressor cells (MDSCs). In some embodiments, the method further comprises administering a colony-stimulating factor 1 receptor (CSF1R).

The subject may be an animal. In some embodiments, the subject is a mammal, an amphibian, or a reptile. The subject may be a mammal. In some embodiments, the mammal is a domesticated animal. For example, the domesticated animal may be selected from a dog, a cat, a cow, a chicken, a pig, a bull, a goat, a sheep, a llama, a horse, a donkey, a camel, a guinea pig, or a bird. In some embodiments, the subject is a human. The human may be diagnosed with or suspected of having a cancer.

In some embodiments the cancer is a primary cancer. In some embodiments, the cancer is a secondary cancer. The cancer may be detected in any organ of the subject. In some embodiments, the cancer is detected in a brain, an eye, a mouth, an esophagus, a lung, a spleen, a pancreas, a liver, a colon, a stomach, an intestine, a reproductive organ such as a prostate, a vagina, or a testicle; or a breast, a bone, a bone marrow, blood, a lymph node, or a patch of skin of the subject. In some embodiments, the cancer is a lung cancer. The lung cancer may be non-small cell lung cancer.

In some embodiments, a pathologist grades the cancer between 0 and 4. The cancer may be a grade 0, a grade 1, a grade 2, a grade 3, or a grade 4. In some embodiments, the cancer is metastatic. In some embodiments, the cancer is non-metastatic. For example, a pathologist would have ordinary skill and be capable of grading a cancer such as non-small cell lung cancer. According to the American Lung Association, describes each grading as follows: Stage 0 or grade 0 is an early-stage lung cancer that is only in the top lining of the lung or bronchus and has not spread; Stage I or grade 1 is divided into two sub-stages, IA and IB, based on the size of the tumor. In Stage I, the cancer has not spread to the lymph nodes or other parts of the body. Stage II or grade 2 is divided into stage IIA and IIB, with each stage then broken into additional sections, depending on: the size of the tumor, where it is found and whether or not the cancer has spread to the lymph nodes. These tumors may be larger than those in stage I and/or have begun to spread to nearby lymph nodes. In stage II, the cancer has not spread to distant organs. Stage III or grade 3 is divided into IIIA, IIIB, or IIIC, depending on the size and location of the tumor and how far it has spread. Most commonly, the cancer has spread to the lymph nodes in the mediastinum (the area in the chest between the lungs). Finally, Stage IV or grade 4 is the most advanced form of NSCLC. In this stage, the cancer has metastasized, or spread, to the lining of the lung or other areas of the body. Doctors determine stage based on tumor size and location, lymph node involvement, and metastasis.

The tumor microenvironment may comprise myeloid derived suppressor cells (MDSCs). The MDSCs are characterized by the expression of CD11c⁺, CD11b⁺, CD13⁺, CD14⁺, CD15⁺, CD33⁺, HLA-DR⁻, or a combination thereof. In some embodiments, the MDSCs are characterized by the expression of CD11b⁺ and CD13⁺. In some embodiments, the MDSCs are characterized by the expression of CD11b⁺ and CD14⁺. In some embodiments, the MDSCs are characterized by the expression of CD11b⁺ and CD15⁺. In some embodiments, the MDSCs are characterized by the expression of CD11b⁺ and CD33⁺. In some embodiments, the MDSCs are characterized by the expression of CD11b⁺, CD13⁺, CD14⁺, HLA-DR⁻, or a combination thereof. The MDSCs may be characterized by CD11b⁺. In some embodiments, the MDSCs may be characterized by the expression of CD13⁺, CD14⁺, or both. In some embodiments, the MDSCs are characterized by the expression of CD11c+ and one or more of CD11b⁺, CD13⁺, CD14⁺, CD15⁺, CD33⁺, or HLA-DR⁻.

The MDSCs may be characterized by a reduced expression level of lysosomal acid lipase (LAL) compared to a myeloid cell not in a tumor microenvironment. In some embodiments, the MDSCs are characterized by an inhibition of LAL expression. In some embodiments, the MDSCs are characterized by an increase expression of PDH compared to a myeloid cell not in a tumor microenvironment. In some embodiments, the MDSCs may be characterized by an increase in expression of PD-L1 compared to a myeloid cell not in a tumor microenvironment.

The composition may be formulated to be administered as a liquid or a powder. In some embodiments, the composition is in the form of a liquid. In some embodiments, the composition is in the form of a powder. In some embodiments, the composition is administered as a pharmaceutical composition or salt thereof. In some embodiments, the composition is formulated based on the location of the tumor microenvironment or the type of cancer.

In some embodiments, the composition is formulated to be administered to a human subject. The pharmaceutical compositions of the present disclosure are suitable for administration to a subject by any suitable means, including without limitation those means used to administer conventional antimicrobials. The pharmaceutical compositions of the present disclosure may be administered using any applicable route that would be considered by one of ordinary skill, including without limitation oral, intravenous (“IV”) injection or infusion, intravesical, subcutaneous (“SC”), intramuscular (“IM”), intraperitoneal, intradermal, intraocular, inhalation (and intrapulmonary), intranasal, transdermal, epicutaneously, subdermal, topical, mucosal, nasal, ophthalmic, impression into skin, intravaginal, intrauterine, intracervical, and rectal. Such dosage forms should allow a compound of the present disclosure to reach target cells. Other factors are well known in the art and include considerations such as toxicity and dosage forms that retard a compound or composition from exerting its effects. Techniques and formulations generally may be found in Remington: The Science and Practice of Pharmacy, 21st edition, Lippincott, Williams and Wilkins, Philadelphia, Pa., 2005.

In some embodiments, the pharmaceutical compositions of the present disclosure are adapted for topical administration. As used herein, the term “topical administration” refers to administration of a compound of the present disclosure to the skin surface of a patient so that a compound of the present disclosure passes through the skin layer. Transdermal administration and transmucosal administration are also encompassed within the term topical administration. As used herein, the term “transdermal” refers to passage of a compound of the present disclosure across at least one skin layer of a subject or patient. As used herein, “transmucosal” refers to passage of a compound of the present disclosure across a mucous membrane of a subject or patient.

In some embodiments, the composition includes a lysosomal acid lipase (LAL) replacement therapy. The LAL replacement therapy may be a recombinant human LAL protein. The LAL replacement therapy may comprise sebelipase alfa or pharmaceutically acceptable salt thereof. Examples of an LAL replacement therapy are disclosed in U.S. Pat. No. 11,235,036, issued Feb. 1, 2022. In some embodiments, the LAL replacement therapy is the commercially available Kanuma.

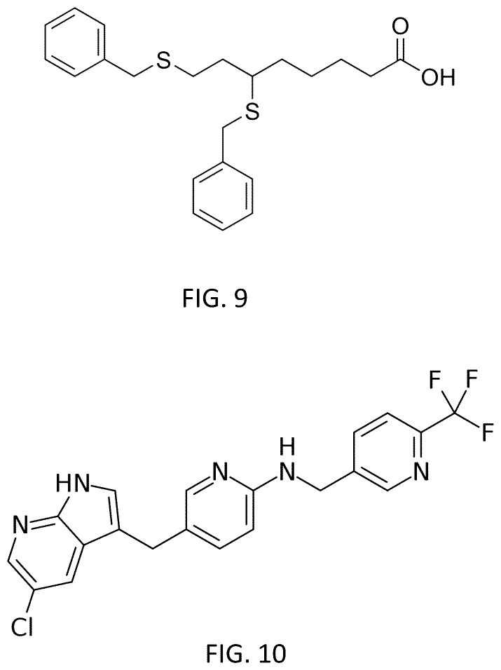

In some embodiments, the composition includes a pyruvate dehydrogenase (PDH) inhibitor. Examples of a PDH inhibitor are disclosed in U.S. 2015/133524 published May 14, 2015. The contents of which are incorporated by reference. In some embodiments, the pyruvate dehydrogenase inhibitor is CPI-613, CAS No.: 95809-78-2, having a chemical structure shown in FIG. 9, or a pharmaceutically acceptable salt thereof, commercially available as DEVIMISTAT.

In some embodiments, the composition comprises a programmed death-ligand 1(PD-L1) inhibitor. Examples of a PD-L1 inhibitor are disclosed in U.S. Pat. No. 11,866,434, issued Jan. 9, 2024. The contents of which are incorporated by reference. In some embodiments, the PD-L1 inhibitor is selected from a commercially small molecule Atezolizumab, Avelumab, or Durvalumab. In some embodiments, the PD-L1 inhibitor is a monoclonal antibody.

In some embodiments, the composition comprises a colony stimulating factor 1 receptor (CSF1R) inhibitor. In some embodiments, the CSF1R inhibitor comprises pexidartinib. Pexidartinib, CAS No.: 1029044-16-3, having a chemical structure shown in FIG. 10, or a pharmaceutically acceptable salt thereof, may be commercially available as the FDA approved drug TURALIO.

The composition may include an LAL replacement therapy and a PDH inhibitor. In some embodiments, the composition comprises an LAL replacement therapy and a PD-L1 inhibitor. In some embodiments, the composition comprises a PDH inhibitor and a PD-L1 inhibitor.

The method may comprise administering an LAL replacement therapy separately from a PDH inhibitor or a PD-L1 inhibitor. For example, the LAL replacement therapy may be administered to a subject orally, while the PDH inhibitor or the PD-L1 inhibitor may be administered to the subject intravenously. In some embodiments, the LAL replacement therapy and the PDH inhibitor or the PD-L1 inhibitor may be both formulated for oral administration to a subject. In some embodiments, a PDH inhibitor is administered to a subject together or separately with the PD-L1 inhibitor. The LAL replacement therapy may be administered at the same time or separately from the PDH inhibitor or the PD-L1 inhibitor.

In some embodiments, the LAL replacement therapy is administered before the PDH inhibitor is administered to the subject. In some embodiments, the LAL replacement therapy is administered after the PDH inhibitor is administered to the subject. In some embodiments, the LAL replacement therapy is administered before the PD-L1 inhibitor. In some embodiments, the LAL replacement therapy is administered after the PD-L1 inhibitor.

In some embodiments, the CSF1R inhibitor is administered before or after the LAL replacement therapy. In some embodiments, the CSF1R inhibitor is administered before or after the PDH inhibitor and/or the PD-L1 inhibitor.

In some embodiments, the composition is administered before, after, or co-administered with an anti-tumor treatment. In some embodiments, the LAL replacement therapy is administered before an anti-tumor treatment. In some embodiments, the LAL replacement therapy is co-administered with the anti-tumor treatment. The LAL replacement therapy may be administered after the administration of an anti-tumor treatment. In some embodiments, the LAL replacement therapy may be administered before and after the administration of an anti-tumor treatment to a subject.

In some embodiments, the PDH inhibitor is administered before an anti-tumor treatment is administered to the subject. In some embodiments, the PDH inhibitor is administered after an anti-tumor treatment is administered to the subject. In some embodiments, the PDH inhibitor is co-administered with the anti-tumor treatment.

In some embodiments, the PD-L1 inhibitor is administered before an anti-tumor treatment. In some embodiments, the PD-L1 inhibitor is administered after the anti-tumor treatment. The method may include co-administering the PD-L1 inhibitor with the anti-tumor treatment.

In some embodiments, the CSF1R inhibitor is administered before an anti-tumor treatment. In some embodiments, the CSF1R inhibitor is administered after the anti-tumor treatment. The method may include co-administering the CSF1R inhibitor with the anti-tumor treatment

The anti-tumor treatment may be any known anti-tumor treatment and will depend on the location and type of cancer being treated. In some embodiments, the anti-tumor treatment is selected from surgery, chemotherapy, radiotherapy, immunotherapy, or a combination thereof.

The immunotherapy may be an antibody or a genetically engineered immune cell, for example a genetically engineered T-cell. The genetically engineered T-cell may be sourced from the subject or another source.

The radiotherapy may be an ionizing radiation selected from high energy x-rays, electron beams, or proton beams.

In some embodiments, the chemotherapy can independently include one or more agents selected from the group consisting of methotrexate, vinblastine, doxorubicin, cisplatin, MVAC (methotrexate, vinblastine, doxorubicin and cisplatin), docetaxel, trastuzumab, cyclophosphamide, paclitaxel, dose-dense AC followed by T (i.e., doxorubicin, cyclophosphamide, paclitaxel), TAC (docetaxel, doxorubicin, cyclophosphamide), fluorouracil, bleomycin, etoposide, vincristine, procarbazine, prednisone, BEACOPP (bleomycin, etoposide, doxorubicin, cyclophosphamide, vincristine, procarbazine, prednisone), gemcitabine, ifosfamide, carboplatin, ICE (ifosfamide, carboplatin, etoposide), rituximab, RICE (rituximab, ifosfamide, carboplatin, etoposide), CHOP-14 (cyclophosphamide, doxorubicin, vincristine, prednisone), mesna, novantrone, MINE (mesna, ifosfamide, novantrone, etoposide), dexamethasone, cytarabine DHAP (dexamethasone, cisplatin, cytarabine), methylprednisolone, ESHAP (etoposide, methylprednisolone, cisplatin, cytarabine), HyperCVAD and rituximab (cyclophosphamide, vincristine, doxorubicin, dexamethasone, rituximab), dacarbazine, vinblastine, dacarbazine-based combination (dacarbazine, cisplatin, vinblastine), dacarbazine-based combination with IL-2 and interferon alfa (dacarbazine, cisplatin, vinblastine, IL-2, interferon alfa), topotecan, MAID (mesna, doxorubicin, ifosfamine, dacarbazine), VeIP (vinblastine, ifosfamide, cisplatin), VIP (etoposide, ifosfamide, cisplatin), TIP (paclitaxel, ifosfamide, cisplatin), gemcitabine, CMF classic (cyclophosphamide, methotrexate, fluorouracil), AC (doxorubicin, cyclophosphamide), FEC (fluorouracil, epirubicin, cyclophosphamide), TC (docetaxel, cyclophosphamide), cisplatin/topotecan, paclitaxel/cisplatin, irincotecan, FOLFOX (fluorouracil, leucovorin, oxaliplatin), irincotecan/cisplatin, epirubicin/cisplatin/5-fluorouracil, epirubicin/cisplatin/capecitabine, DT-PACE (dexamethasone/thalidomide/cisplatin/doxorubicin/cyclophosphamide/etoposide), ET-PACE and bortezomib, EPOCH (etoposide, prednisone, vincristine, cyclophosphamide, doxorubicin), GDP (gemcitabine, dexamethasone, cisplatin), GDP and rituximab, FMR (fludarabine, mitoxantrone, rituximab, CHOP and rituximab (cyclophosphamide, doxorubicin, vincristine, prednisone, rituximab), cisplatin/paclitaxel, cisplatin/vinorelbine, cisplatin/docetaxel, ciaplatin/etoposide, carboplatin/paclitaxel, carboplatin/docetaxel, FOLFIRINOX (5-FU/leucovorin, irinotecan and oxaliplatin), cabazitaxel, etoposide/carboplatin, etoposide/cisplatin. In some embodiments, the chemotherapy can independently include one or more agents selected from the group consisting of methotrexate, vinblastine, doxorubicin, cisplatin, docetaxel, trastuzumab, cyclophosphamide, paclitaxel, fluorouracil, bleomycin, etoposide, vincristine, procarbazine, prednisone, gemcitabine, ifosfamide, carboplatin, mesna, novantrone, cytarabine methylprednisolone, rituximab dacarbazine, vinblastine, topotecan, gemcitabine, irincotecan, epirubicin, 5-fluorouracil, capecitabine, bortezomib, and cabazitaxel.

In some embodiments, the chemotherapy can include one or more agents selected from the group consisting of methotrexate, vinblastine, doxorubicin, cisplatin, MVAC (methotrexate, vinblastine, doxorubicin and cisplatin), trastuzumab, cyclophosphamide, dose-dense AC followed by T (i.e., doxorubicin, cyclophosphamide, paclitaxel), fluorouracil, bleomycin, etoposide, vincristine, procarbazine, prednisone, BEACOPP (bleomycin, etoposide, doxorubicin, cyclophosphamide, vincristine, procarbazine, prednisone), gemcitabine, ifosfamide, carboplatin, ICE (ifosfamide, carboplatin, etoposide), rituximab, RICE (rituximab, ifosfamide, carboplatin, etoposide), CHOP-14 (cyclophosphamide, doxorubicin, vincristine, prednisone), mesna, novantrone, MINE (mesna, ifosfamide, novantrone, etoposide), dexamethasone, cytarabine DHAP (dexamethasone, cisplatin, cytarabine), methylprednisolone, ESHAP (etoposide, methylprednisolone, cisplatin, cytarabine), HyperCVAD and rituximab (cyclophosphamide, vincristine, doxorubicin, dexamethasone, rituximab), dacarbazine, vinblastine, dacarbazine-based combination (dacarbazine, cisplatin, vinblastine), dacarbazine-based combination with IL-2 and interferon alfa (dacarbazine, cisplatin, vinblastine, IL-2, interferon alfa), topotecan, MAID (mesna, doxorubicin, ifosfamine, dacarbazine), VeIP (vinblastine, ifosfamide, cisplatin), VIP (etoposide, ifosfamide, cisplatin), TIP (paclitaxel, ifosfamide, cisplatin). In some embodiments, the gemcitabine, CMF classic (cyclophosphamide, methotrexate, fluorouracil), AC (doxorubicin, cyclophosphamide), FEC (fluorouracil, epirubicin, cyclophosphamide), cisplatin/topotecan, paclitaxel/cisplatin, irincotecan, FOLFOX (fluorouracil, leucovorin, oxaliplatin), irincotecan/cisplatin, epirubicin/cisplatin/5-fluorouracil, epirubicin/cisplatin/capecitabine, DT-PACE (dexamethasone/thalidomide/cisplatin/doxorubicin/cyclophosphamide/etoposide), ET-PACE and bortezomib, EPOCH (etoposide, prednisone, vincristine, cyclophosphamide, doxorubicin), GDP (gemcitabine, dexamethasone, cisplatin), GDP and rituximab, FMR (fludarabine, mitoxantrone, rituximab, CHOP and rituximab (cyclophosphamide, doxorubicin, vincristine, prednisone, rituximab), cisplatin/paclitaxel, cisplatin/vinorelbine, ciaplatin/etoposide, carboplatin/paclitaxel, FOLFIRINOX (5-FU/leucovorin, irinotecan and oxaliplatin), cabazitaxel, etoposide/carboplatin, etoposide/cisplatin. In some embodiments, the chemotherapy can include one or more agents selected from the group consisting of methotrexate, vinblastine, doxorubicin, cisplatin, trastuzumab, cyclophosphamide, fluorouracil, bleomycin, etoposide, vincristine, procarbazine, prednisone, gemcitabine, ifosfamide, carboplatin, mesna, novantrone, cytarabine methylprednisolone, rituximab dacarbazine, vinblastine, topotecan, gemcitabine, irincotecan, epirubicin, 5-fluorouracil, capecitabine, and bortezomib.

The methods disclosed herein comprise administering the composition to a subject before administering the anti-tumor treatment. In some embodiments, the composition is administered after the anti-tumor treatment. In some embodiments, the composition is administered before or after the anti-tumor treatment is administered. In some embodiments, the composition is administered before and after the anti-tumor treatment is administered. In some embodiments, the composition is co-administered with the anti-tumor treatment.

The administered dosage of the composition and the dosage of the anti-tumor treatment will be dependent on the type and location of the cancer. A person having ordinary skill in the art will be able ascertain an initial dosage, and the dosage may be adjusted over the course of the treatment of the cancer. In some embodiments, the composition is administered at least once a day. In some embodiments, the composition is administered at least twice a day. The composition may be administered for at least a week. In some embodiments, the composition is administered for at least two weeks.

In some embodiments, the method comprises inhibiting a tumor microenvironment comprising administer a composition, wherein the composition includes and LAL replacement therapy, and administering an anti-tumor treatment, wherein the anti-tumor treatment is an immunotherapy.

In addition to the aspects and embodiments described and provided elsewhere in the present disclosure, the following non-limiting list of embodiments are also contemplated.
Clause 1. A method of treating a cancer in a subject comprising: inhibiting a tumor microenvironment comprising administering a therapeutically effective amount of a composition including an LAL replacement therapy, a PDH inhibitor, a PD-L1 inhibitor, or a combination thereof; and administering an anti-tumor treatment.
Clause 2. A method of inhibiting a tumor microenvironment in a subject comprising administering a therapeutically effective amount of a composition including an LAL replacement therapy, a PDH inhibitor, a PD-L1 inhibitor, or a combination thereof.
Clause 3. The embodiment of clauses 1 and 2, wherein the subject is an animal.
Clause 4. The embodiment of clauses 1 and 3, wherein the subject is a mammal.
Clause 5. The embodiment of clauses 1 to 4, wherein the subject is a domesticated animal selected from a dog, a cat, a cow, a chicken, a pig, a bull, a goat, a sheep, a llama, a horse, a donkey, a camel, a guinea pig, or a bird.
Clause 6. The embodiment of clauses 1 to 4, wherein the subject is a human.
Clause 7. The embodiment of clauses 1 to 6, wherein the cancer is a primary cancer.
Clause 8. The embodiment of clauses 1 to 6, wherein the cancer is a secondary cancer.
Clause 9. The embodiment of clauses 1 to 8, wherein the cancer is detected in a brain, an eye, a mouth, an esophagus, a lung, a spleen, a pancreas, a liver, a colon, a stomach, an intestine, a prostate, a vagina, a breast, a bone, a bone marrow, a blood, a testicle, a lymph node, or a skin of the subject.
Clause 10. The embodiment of clauses 1 to 9, wherein the cancer is a lung cancer.
Clause 11. The embodiment of clauses 1 to 10, wherein the cancer is a non-small cell lung cancer.
Clause 12. The embodiment of clause 1 to 11, wherein the cancer is a grade 0.
Clause 13. The embodiment of clauses 1 to 11, wherein the cancer is a grade 1.
Clause 14. The embodiment of clauses 1 to 11, wherein the cancer is a grade 2.
Clause 15. The embodiment of clauses 1 to 11, wherein the cancer is a grade 3.
Clause 16. The embodiment of clauses 1 to 11, wherein the cancer is a grade 4.
Clause 17. The embodiment of clauses 1 to 16, wherein the cancer is metastatic.
Clause 18. The embodiment of clauses 1 to 17, wherein the tumor microenvironment includes myeloid derived suppressor cells (MDSCs).
Clause 19. The embodiment of clauses 1 to 18, wherein the MDSCs are characterized by the expression of CD11c⁺, CD11b⁺, CD13⁺, CD14⁺, CD15⁺, CD33⁺, HLA-DR⁻, or a combination thereof.
Clause 20. The embodiment of any of the relevant previous clauses, wherein the MDSCs are characterized by the expression of CD11b⁺ and CD13⁺.
Clause 21. The embodiment of any of the relevant previous clauses, wherein the MDSCs are characterized by the expression of CD11b⁺ and CD14⁺.
Clause 22. The embodiment of any of the relevant previous clauses, wherein the MDSCs are characterized by the expression of CD11b⁺ and CD15⁺.
Clause 23. The embodiment of any of the relevant previous clauses, wherein the MDSCs are characterized by the expression of CD11b⁺ and CD33⁺.
Clause 24. The embodiment of any of the relevant previous clauses, wherein the MDSCs are characterized by the expression of CD11b⁺, CD13⁺, CD14⁺, HLA-DR⁻, or a combination thereof.
Clause 25. The embodiment of any of the previous relevant clauses, wherein the MDSCs are characterized by a reduced expression level of lysosomal acid lipase (LAL) compared to a myeloid cell not in a tumor microenvironment.
Clause 26. The embodiment of any of the previous relevant clauses, wherein the MDSCs are characterized by an inhibition of LAL expression.
Clause 27. The embodiment of any of the previous relevant clauses, wherein the composition is in the form of a liquid.
Clause 28. The embodiment of any of the previous relevant clauses, wherein the composition is in the form of a powder.
Clause 29. The embodiment of any of the previous relevant clauses, wherein the composition is a pharmaceutical composition or salt thereof.
Clause 30. The embodiment of any of the previous relevant clauses, wherein the composition includes a lysosomal acid lipase (LAL) replacement therapy.
Clause 31. The embodiment of any of the previous relevant clauses, wherein the LAL replacement therapy comprises sebelipase alfa.
Clause 32. The embodiment of any of the previous relevant clauses, wherein the LAL replacement therapy is the commercially available Kanuma.
Clause 33. The embodiment of any of the previous relevant clauses, wherein the composition includes a PDH inhibitor.
Clause 34. The embodiment of any of the relevant clauses, wherein the composition includes a PD-L1 inhibitor.
Clause 35. The embodiment of any of the relevant clauses, wherein the composition includes an LAL replacement therapy and a PDH inhibitor.
Clause 36. The embodiment of any of the relevant clauses, wherein the composition includes an LAL replacement therapy and a PD-L1 inhibitor.
Clause 37. The embodiment of any of the relevant clauses, wherein the composition includes a PDH inhibitor and a PD-L1 inhibitor.
Clause 38. The embodiment of any of the relevant clauses, wherein the composition is administered intravenously, orally, topically, or through inhalation.
Clause 39. The embodiment of any of the relevant clauses, wherein the LAL replacement therapy is administered before the PDH inhibitor is administered to the subject.
Clause 40. The embodiment of any of the relevant clauses, wherein the LAL replacement therapy is administered before the PD-L1 therapy.
Clause 41. The embodiment of any of the relevant clauses, wherein the LAL replacement therapy is administered before the anti-tumor treatment.
Clause 42. The embodiment of any of the relevant clauses, wherein the LAL replacement therapy is co-administered with the anti-tumor treatment.
Clause 43. The embodiment of any of the relevant clauses, wherein the PHD inhibitor is administered before the anti-tumor treatment is administered to the subject.
Clause 44. The embodiment of any of the relevant clauses, wherein the PHD inhibitor is co-administered with the anti-tumor treatment.
Clause 45. The embodiment of any of the relevant clauses, wherein PD-L1 inhibitor is administered before the anti-tumor treatment.
Clause 46. The embodiment of any of the relevant clauses, wherein PD-L1 inhibitor is co-administered with the anti-tumor treatment.
Clause 47. The embodiment of any of the relevant clauses, wherein the anti-tumor treatment is selected from surgery, chemotherapy, radiotherapy, immunotherapy, or a combination thereof.
Clause 48. The embodiment of any of the relevant clauses, wherein the anti-tumor treatment includes surgery,
Clause 49. The embodiment of any of the relevant clauses, wherein the anti-tumor treatment includes chemotherapy.
Clause 50. The embodiment of any of the relevant clauses, wherein the anti-tumor treatment includes radiotherapy.
Clause 51. The embodiment of any of the relevant clauses, wherein the anti-tumor treatment includes immunotherapy.
Clause 52. The embodiment of any of the relevant clauses, wherein the anti-tumor treatment includes a genetically modified T-cell.
Clause 53. The embodiment of any of the relevant clauses, wherein the composition is administered before the anti-tumor treatment.
Clause 54. The embodiment of any of the relevant clauses, wherein the composition is administered after the anti-tumor treatment.
Clause 55. The embodiment of any of the relevant clauses, wherein the composition is administered before or after the anti-tumor treatment is administered.
Clause 56. The embodiment of any of the relevant clauses, wherein the composition is administered before and after the anti-tumor treatment is administered.
Clause 57. The embodiment of any of the relevant clauses, wherein the composition is administered at least once a day.
Clause 58. The embodiment of any of the relevant clauses, wherein the composition is administered at least twice a day.
Clause 59. The embodiment of any of the relevant clauses, wherein the composition is administered for at least a week.
Clause 60. The embodiment of any of the relevant clauses, wherein the composition is administered for at least two weeks.
Clause 61. The embodiment of any of the relevant clauses, wherein the composition includes and LAL replacement therapy and the anti-tumor treatment is an immunotherapy.

Further reference to the methods and compositions are made to the following experimental examples.
Clause 62. The embodiment of any of the relevant clauses, wherein the method further comprises administering a CSF1R inhibitor.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention. Changes therein and other uses which are encompassed within the spirit of the invention as defined by the scope of the claims will occur to those skilled in the art.

### General Methods

Animals and Cell Lines Wild type (Lal^{+/+}, Lal expressed) and Lal^{-/-} (knockout of Lal) mice of the Friend Virus B/NIH (FVB/N) background were bred in house. Both male and female mice aged three-to five-month-old were used, and all the mice have been backcrossed for more than 10 generations. The murine B16 melanoma cell line and Lewis lung carcinoma (LLC) cell line were acquired from American Type Culture Collection. (ATCC, Manassas, VA) Both cell lines were cultured in Dulbecco's Modified Eagle Medium (DMEM) supplemented with 10% Fetal Bovine Serum (FBS) in a 37° C. incubator with 5% CO₂. Cell media was of the Gibco, cell media type, variety and acquired from ThermoFisher Scientific.

Human Blood Samples The blood donor characteristics of the human blood samples of normal subjects and non-small-cell lung cancer (NSCLC, at stage III-IV) patients is described in TABLE 8. NSCLC patients were selected by screening with Programmed death-ligand 1 (PD-L1) Tumor Proportion Score (TPS)≥10%. Both normal subjects and NSCLC patients include male and female, White and African American. To inhibit lysosomal acid lipase (LAL) activity, human blood cells from healthy participants had the red blood cells (RBCs) removed with lysis buffer acquired from Biolegend (San Diego, CA); washed with phosphate buffered saline (PBS) by centrifugation at 240×g for 5 minutes at room temperature; and then incubated with 10 μM LAL inhibitor Lalistat2, acquired from Cayman, Ann Arbor, MI) for 24 hours. As a control, human blood cells were incubated with dimethyl sulfoxide (DMSO).

Isolation of Bone Marrow Lymphocyte Antigen 6 Member G—Positive (Ly6G⁺) Cell Ly6G⁺ cells were isolated by magnetic bead sorting. For CPI-613 (also known as Devimistat; a pyruvate dehydrogenase inhibitor) treatment, freshly isolated Ly6G⁺ cells were pre-treated with DMSO or 10 μM CPI-613 at 37° C. for 1 hour, and then co-cultured with Cluster of Differentiation 4 Protein-positive white blood cells (CD4⁺ T cells) or co-injected with B16 melanoma cells for further analysis.

Single-Cell Ribonucleic Acid (RNA) Sequencing (scRNA-seg) and Data Analysis Ly6G⁺ cells were sorted from the bone marrow of Lal^{+/+} and Lal^{-/-} mice. To obtain equal amount of cell numbers, cells sorted from 6 Lal^{+/+} mice and 6 Lal^{-/-} mice were pooled together and mixed well. The number and viability of Ly6G⁺ cells were 1100 cells/μL and >95%, respectively. Immediately after sorting, Ly6G⁺ single cells were run on the 10×Chromium, single cell assay analysis from 10×Genomics, and then through library preparation following the recommended protocol for the Chromium Single Cell 3′ Reagent Kit, assay to measure single cell transcriptome gene expression. Libraries were run on the NovaSeq S1 for Illumina sequencing, transcriptome sequencing. CellRanger 3.0.2, analyzing and visualizing software, was utilized to process the raw sequence data generated and data were analyzed as previously described.

Western Blot Analysis Western blot analysis was performed using antibodies against hexokinase 1 (HK1), hexokinase 2 (HK2), hexokinase 3 (HK3), glucose-6-phosphate dehydrogenase (G6PD), pyruvate dehydrogenase (PDH), lactate dehydrogenase A (LDHA), lactate dehydrogenase B (LDHB), and glutamate dehydrogenase (GLUD) (rabbit monoclonal antibodies; used at a ratio of 1:1000; acquired from Cell Signaling). Antibody against β-actin (rabbit monoclonal antibody; used at a ratio of 1:1000; acquired from Cell Signaling) was used as a loading control. For detection, the membrane was incubated with anti-rabbit immunoglobin G (IgG) secondary antibodies conjugated with horseradish peroxidase (used at a ratio of 1:2000; acquired from Cell Signaling). Bands were visualized using SuperSignal West Pico Chemiluminescent substrate, chemiluminescent detection kit acquired from ThermoScientific Pierce.

Measurement of Glucose, Pyruvate and α-ketoglutarate Levels Measurement of glucose, pyruvate and α-ketoglutarate levels was performed using the glucose assay kit (Sigma, cat #GAHK20), pyruvate assay kit (Sigma, cat #MAK071) and a-ketoglutarate assay kit (Sigma, cat #MAK054), respectively. Briefly, samples were prepared from freshly isolated Ly6G⁺ cells according to assay kits' instructions. Lysates were then incubated with reaction mix for a designated time, and ready for the absorbance measuring.

Flow Cytometry Analysis For analyses of percentages of CD11b⁺Ly6G⁺ (integrin alpha M-positive/Ly6G positive) and Ly6G⁺Ly6C⁺ (Ly6G positive/lymphocyte antigen 6 C1-positive) cells, single cells harvested from the bone marrow of Lal^{+/+} and Lal^{-/-} mice were stained with APC eFluor 780-conjugated, dye, anti-Ly6G antibody (1A8-Ly6g, cat #47-9668-82), FITC (fluorescein isothiocyanate)-conjugated anti-CD11b antibody (M1/70, cat #11-0112-82), and PE (phycocrythrin)-conjugated anti-Ly6C antibody (HK1.4, cat #12-5932-82) at 4° C. for 15 minutes. All antibodies were acquired from eBioscience, San Diego, CA. For analysis of PD-L1 expression in Ly6G⁺ cells, cells were stained with APC eFluor 780-conjugated anti-Ly6G antibody, PE-conjugated anti-CD11c (integrin alpha X) antibody (N418, cat #12-0114-82) (acquired from eBioscience), and PE-Cy7 (cyanine 7)-conjugated anti-PD-L1 antibody (10F.9G2, cat #124314) (acquired from Biolegend) at 4° C. for 15 minutes. Cells were washed with PBS, then were ready for flow cytometry analysis.

For flow cytometry analysis of human blood samples, human blood cells had the RBCs removed, washed with PBS, and stained with APC-eFluor 780-conjugated anti-CD11b antibody (ICRF44, cat #47-0118-42), FITC-conjugated anti-CD13 (aminopeptidase N) antibody (WM15, cat #11-0138-42), PE-conjugated anti-CD14 antibody (61D3, cat #12-0149-42), APC-conjugated anti-CD15 (Lewis X) antibody (MMA, cat #17-0158-42), PE-conjugated anti-CD33 antibody (HIM3-4, cat #12-0339-42), PE-Cy7-conjugated anti-HLA-DR (major histocompatibility complex, class II) antibody (L243, cat #25-9952-42) (all acquired from eBioscience) at 4° C. for 15 minutes. Cells were then washed with PBS, fixed with 1% paraformaldehyde, and prepared for flow cytometry analysis. To analyze LAL levels, cells were further fixed and permeabilized using BD Cytofix/Cytoperm Fixation/Permeabilization Kit, cell fixation and permeabilization kit, and incubated with non-fluorescence conjugated anti-LAL antibody at 4° C. overnight. To analyze metabolic enzyme levels, cells after fixation and permeabilization were incubated with non-fluorescence conjugated antibodies against metabolic molecules including G6PD (D5D2, cat #12263S), LDH (C28H7, cat #3558S), PDH (cat #2784S), and GLUD (D9F7P, cat #12793S) (antibodies acquired from Cell Signaling, Beverly, MA) at 4° C. overnight. On the next day, cells were washed and stained with APC-or FITC-conjugated anti-rabbit IgG antibody at 4° C. for 30 minutes, then washed for flow cytometry analysis. For flow cytometry analysis, ≥50,000 cells were acquired and scored using a LSRII machine, flow cytometer machine, (for mouse samples) or Fortessa, flow cytometer machine, (for human samples) (acquired from BD Biosciences). Data were processed using BD CellQuest Pro software (version 19.f3fcb), flow cytometer data analysis software, and FlowJo (version 10.6.1), flow cytometer data analysis software (acquired from BD Biosciences).

T cell Proliferation Assay CD4⁺ T cells were isolated from the spleen and labeled with carboxyfluorescein succinimidyl ester (CFSE) as previously described. CFSE-labeled CD4⁺ T cells were co-cultured with Ly6G⁺ cells in 96-well plates pre-coated with anti-CD3 mAb (2 μg/mL) (145-2C11, cat #553057) and anti-CD28 mAb (5 μg/mL) (37.51, cat #553295) (acquired from BD Biosciences) at 37° C., 5% CO₂, for 4 days. The ratio of Ly6G⁺ cells to CD4⁺ T cells was 1:5. Proliferation of CD4⁺ T cells was evaluated as CFSE dilution by flow cytometry analysis.

Reactive Oxygen Species (ROS) Measurement The ROS level in Ly6G⁺ cells was measured by flow cytometry as previously described. Ly6G⁺ cells from Lal^{+/+} and Lal^{-/-} mice with or without CPI-613 pre-treatment were collected and stained with APC eFluor 780-conjugated anti-Ly6G antibody, FITC-conjugated anti-CD11b antibody and 2 μmol/L 2′, 7′-dichlorofluorescein diacetate (acquired from Invitrogen, Carlsbad, CA) at 37° C. for 30 minutes. After PBS washing, the ROS level in Ly6G⁺ cells was analyzed using an LSRII machine.

Subcutaneous Injection of Tumor Cells into Lal^{+/+} Mice To study the effect of Ly6G⁺ cells on tumor growth, isolated 1×10⁶ Ly6G⁺ cells from Lal^{+/+} or Lal^{-/-} mice were pre-treated with DMSO or 10 μM CPI-613 for 1 hour, mixed with 2×10⁵ B16 melanoma cells, and the cell mixture was injected subcutaneously at the flank region of Lal^{+/+} recipient mice. The tumor growth was monitored twice a weck. The tumor volume (mm³) was estimated by measuring the maximal length (L) and width (W) of a tumor and calculated using the formula: (length×width²)/2. For tumor-bearing myeloid-derived suppressor cells (MDSCs) experiments, Lal^{+/+} or Lal^{-/-} mice were injected with 1×10⁶ B16 melanoma cells at flank sites on two sides. Fourteen days later, the mice were sacrificed. Tumor tissues were harvested, digested for single cell preparation, and stained for flow cytometry analysis of the CD11b⁺Ly6G⁺ cells.

In Vitro Co-Culture of Ly6G⁺ and Tumor Cells B16 melanoma or LLC cells were harvested, resuspended, and adjusted to density at 5×10⁴ cells/mL. Isolated Ly6G⁺ cells with or without 10 μM CPI-613 pre-treatment were prepared at the cell density of 5×10⁶ cells/mL. One hundred microliter of Ly6G⁺ cells and 100 μL of B16 melanoma/LLC cells were mixed, and seeded into a well of 96-well plates in DMEM supplemented with 10% FBS. Seventy-two hours later, unattached Ly6G⁺ cells were removed by washing with PBS, and the number of attached B16 melanoma/LLC cells was counted. Morphologically, Ly6G⁺ cells are much smaller than B16 melanoma/LLC cells for exclusion.

Small Interfering RNA Transfection Before transfection, Ly6G⁺ cells were seeded into 96-well plates at a density of 1×10⁶ cells/well. For small interfering RNA (siRNA)-mediated gene knockdown, 50 nmol/L of PDH siRNA (containing a mixture of three independent siRNAs targeting different regions of PDH) or control siRNA was transfected into Ly6G⁺ cells with DharmaFECT Transfection Reagent I, chemical formulation for transfection, (acquired from Dharmacon) according to the manufacturer's protocol. After 24 hours of transfection, cells were harvested for further analysis.

Statistics Data are expressed as mean±SD. Differences between two treatment groups were compared by 2-tailed Student's t-test. When more than two groups were compared, one-way ANOVA with post-hoc Newman-Keul's multiple comparison test was used. When the data were entered into a Grouped table with subcolumns, two-way ANONA with multiple comparison test was used. A P value less than 0.05 was considered statistically significant. All analyses were performed with GraphPad Prism 8.4.1, statistical analysis program (acquired from GraphPad. San Diego, CA).

### Example 1 Characteristics of Lal^{-/-} Versus Lal^{+/+} Ly6G⁺ Cells by scRNA-seq

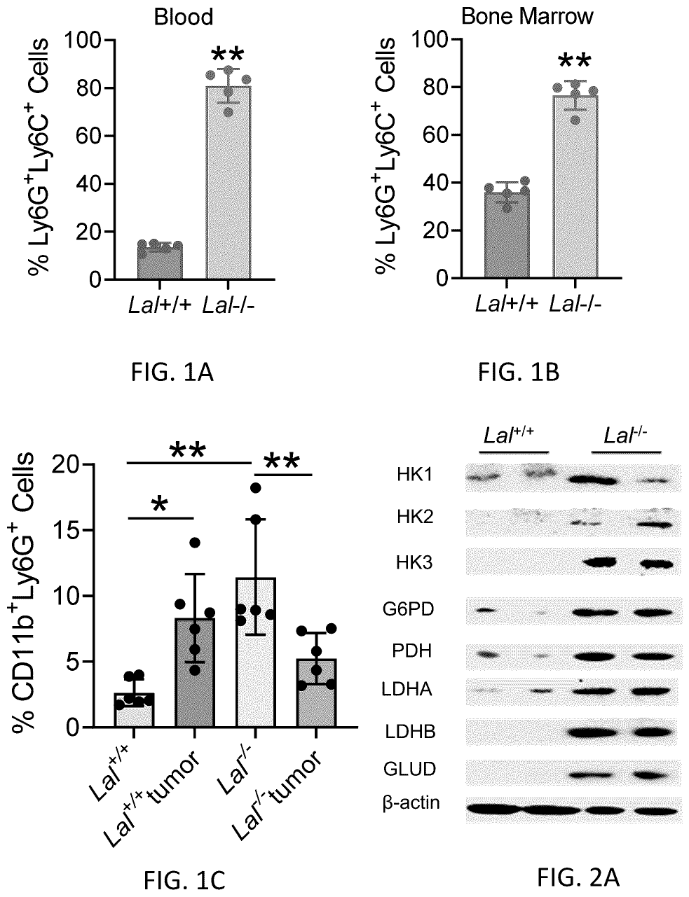

Unlike classical monocytic (M)-MDSCs (CD11b⁺Ly6C⁺) and polymorphonuclear (PMN)-MDSCs (CD11b⁺Ly6G⁺) in tumor-infiltrating MDSCs, LAL-D (deficiency of LAL) induced MDSCs are both Ly6G high and Ly6C high (>76-80% double positive) in the blood and bone marrow (FIG. 1A and FIG. 1B). Collectively, they are defined as LAL-D MDSCs and purified using Ly6G antibody. In comparison to blood and bone marrow LAL-D MDSCs, tumor-infiltrating CD11b⁺Ly6G⁺ cells were not increased in Lal^{-/-} mice after tumor transplantation, which is probably due to over-burden and pool exhaustion as the myeloid compartment is already significantly expanded (FIG. 1C). Since the bone marrow LAL-D CD11b⁺Ly6G⁺ cells are the source for MDSCs expansion, which have been reported to have immunosuppressive and tumor-stimulatory functions to facilitate tumor growth and metastasis, they are chosen for further analyses. To better reveal the underlying mechanisms and get more comprehensive understanding of Lal^{-/-} versus Lal^{+/+} Ly6G⁺ cells, scRNA-seq was performed. Ly6G⁺ cells were isolated from the bone marrow of Lal^{+/+} and Lal^{-/-} mice for scRNA-seq analysis. The volcano plot of gene differential expression and the heat maps of top 50 upregulated and downregulated genes were identified. Further analysis of the T-stochastic neighbor embedding (tSNE) plot identified two major distinctive cellular clusters of Ly6G⁺ cells: (a) clusters 1, 2, 3 (referred to as cluster 123 hereafter) with increased cellular numbers, and (b) clusters 0, 4, 6, 8 (referred to as cluster 0468 hereafter) with decreased cellular numbers in Lal^{-/-} Ly6G⁺ cells versus Lal^{+/+} Ly6G⁺ cells. Both cluster 123 and cluster 0468 demonstrated the neutrophil feature. The top upregulated genes and downregulated genes in cluster 123 and cluster 0468 of Lal^{-/-} Ly6G⁺ cells can be categorized into three groups: (a) those expressed by an increased number of cells in cluster 123 with no change in cluster 0468; (b) those expressed by an increased number of cells in cluster 123 and a decreased number in cluster 0468; and (c) those expressed by an increased number of cells in both cluster 123 and cluster 0468. Log fold change, gene expression, and cellular numbers of the top 50 upregulated and downregulated genes among all clusters, cluster 123, and cluster 0468 were noted for further analysis.

### Example 2 Metabolic Reprogramming in Lal^{-/-} Ly6G⁺ Cells

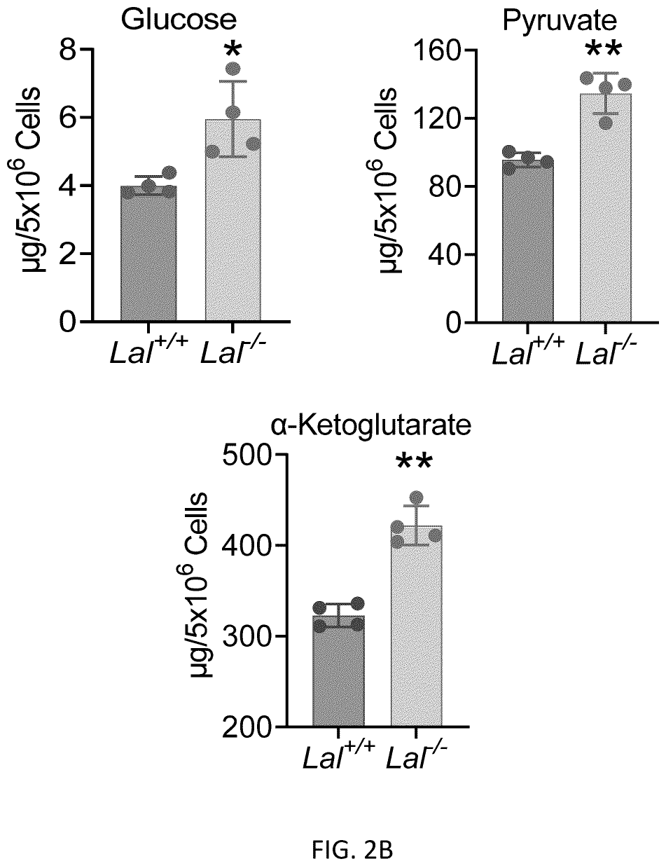

During pathway analysis of scRNA-seq, we noticed that genes involved in glycolysis and the citrate cycle were upregulated in cluster 123 and downregulated in cluster 0468 of Lal^{-/-} Ly6G⁺ cells versus Lal^{+/+} Ly6G⁺ cells. Similarly, when expression of genes responding to ROS (selected from the Mouse Genome Informatics (MGI) database) was compared between these two clusters, there was a significant shift from cluster 0468 to cluster 123, with increased expression in cluster 123 of Lal^{-/-} Ly6G⁺ cells versus Lal^{+/+} Ly6G⁺ cells. The above observations were confirmed by Western blot assay, in which protein expressions of HK1, HK2, HK3 and PDH in the glycolytic pathway, G6PD in the pentose phosphate pathway, and LDH in the anaerobic glycolysis were all increased in Lal^{-/-} Ly6G⁺ cells. Additionally, the concentrations of glucose (the entry substrate of glycolysis) and pyruvate (the end product of glycolysis) were measured in bone marrow Ly6G⁺ cells. Results showed that both glucose flux and pyruvate level were increased in Lal^{-/-} Ly6G⁺ cells versus Lal^{+/+} Ly6G⁺ cells, an indication of increased glycolysis. GLUD in the glutamine pathway also showed upregulation in Lal^{-/-} Ly6G⁺ cells versus Lal^{+/+} Ly6G⁺ cells at the protein level, accompanied with increased α-ketoglutarate level (FIG. 2A and FIG. 2B). Taken together, these results suggest a metabolic switch to glucose and amino acid utilization in Lal^{-/-} Ly6G⁺ cells.

### Example 3 Inhibition of PDH Reversed Overproduction of ROS, Suppression of T Cell Proliferation and Stimulation of Tumor Cell Proliferation in Lal^{-/-} Ly6G⁺ Cells

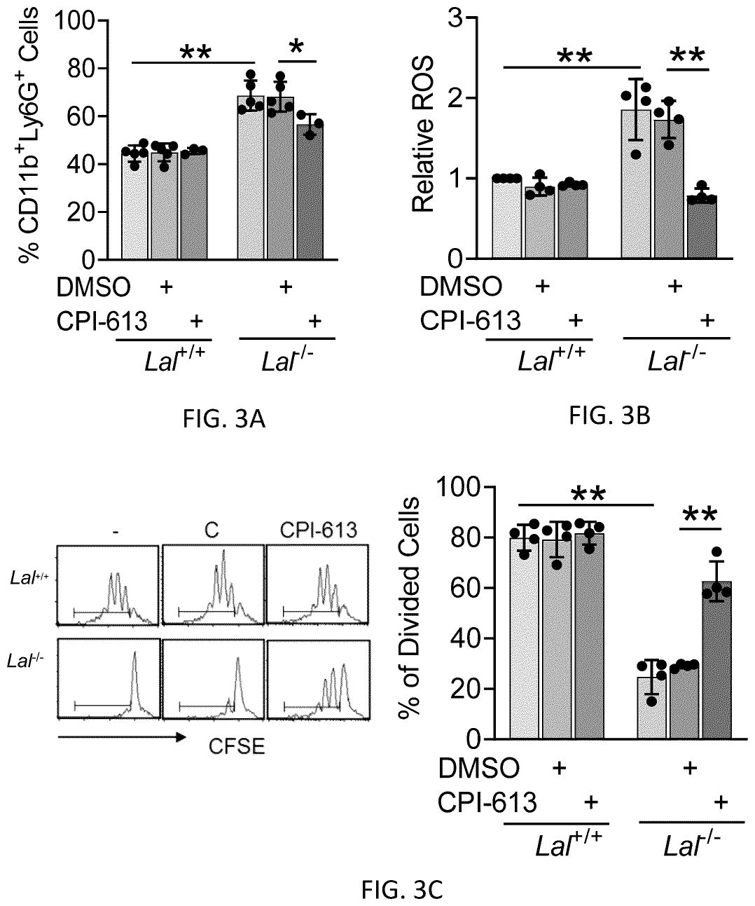

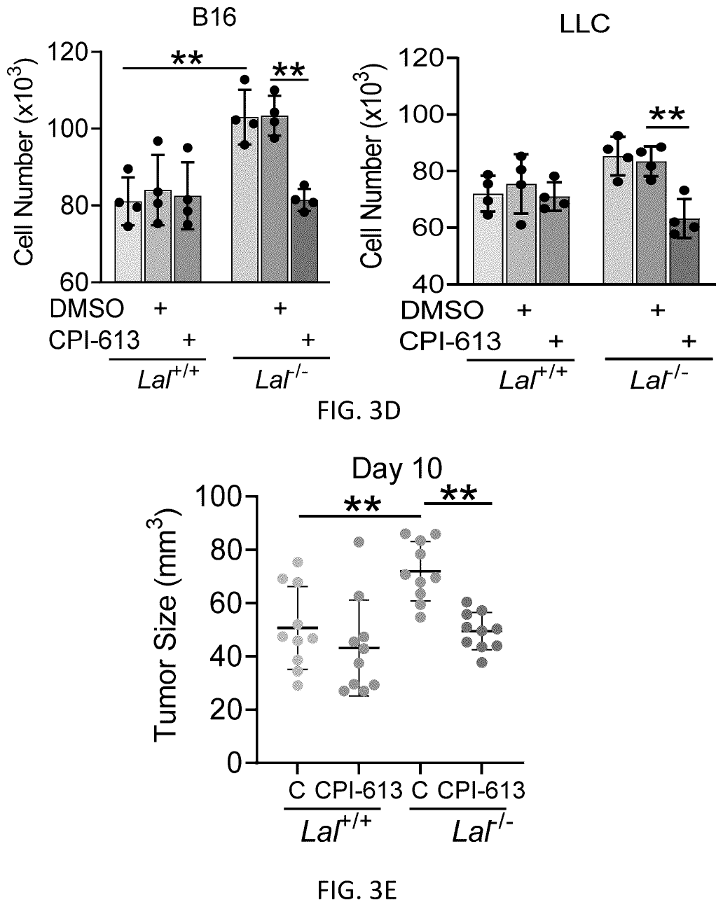

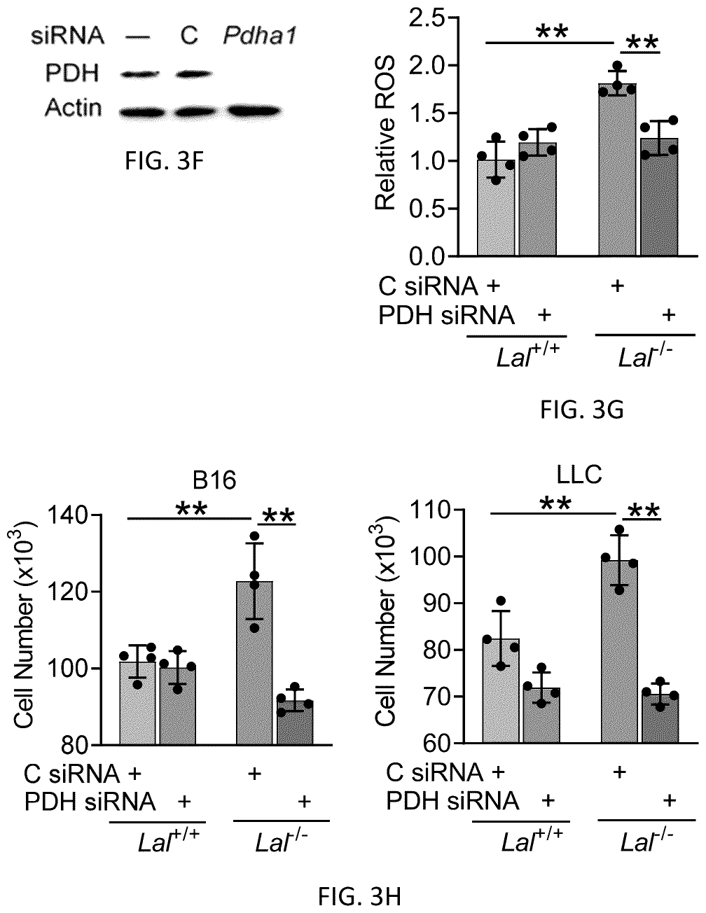

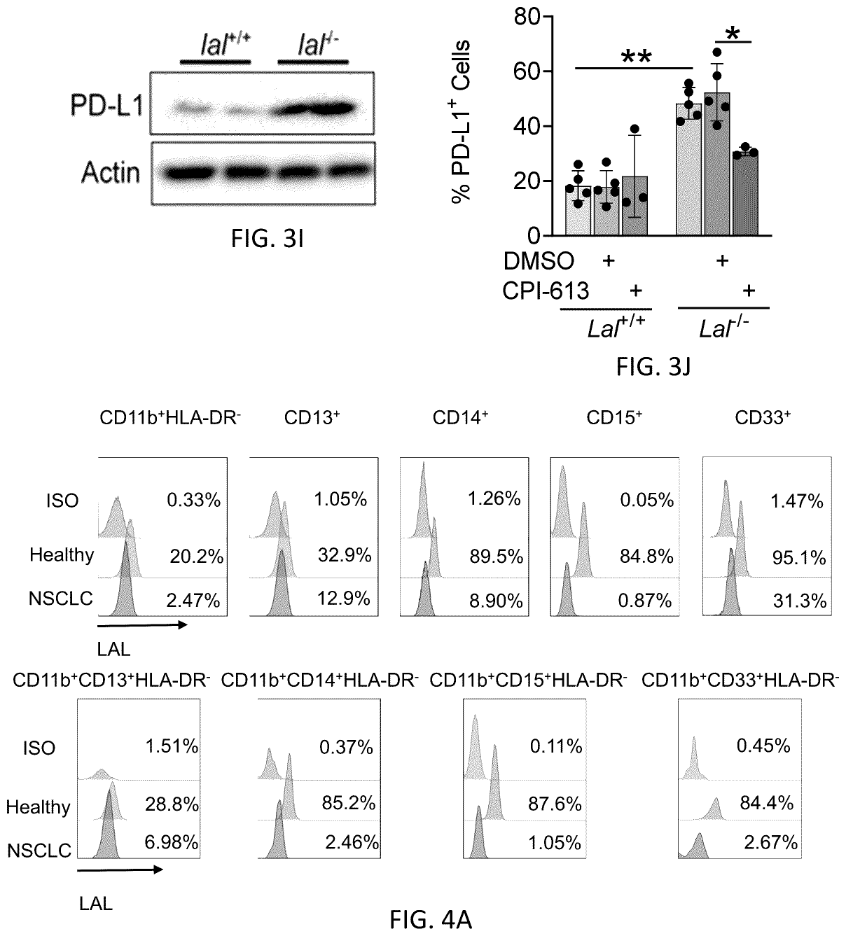

PDH is a key mitochondrial enzyme that acts as the entry point for pyruvate entering the citric acid cycle (TCA cycle) by transforming into acetyl coenzyme A (acetyl-CoA), whose expression was upregulated in Lal^{-/-} Ly6G⁺ cells versus Lal^{+/+} Ly6G⁺ cells (FIG. 2A). To assess the functional relevance of PDH upregulation, PDH inhibitor CPI-613 was used to block glycolysis in Lal^{-/-} Ly6G⁺ cells. Injection of CPI-613 into mice reduced Lal^{-/-} CD11b⁺Ly6G⁺ cells versus CD11b⁺Ly6G⁺ cells (FIG. 3A). In addition, CPI-613 significantly reversed the overproduction of ROS in Lal^{-/-} CD11b⁺Ly6G⁺ cells versus Lal^{+/+} Ly6G⁺ cells (FIG. 3B). The effect of PDH inhibition on Lal^{-/-} Ly6G⁺ cells' functions was further investigated. To evaluate if CPI-613 treatment of Lal^{-/-} Ly6G⁺ cells affect suppression on T cells, CFSE-labeled T cells were co-cultured with Lal^{+/+} or Lal^{-/-} Ly6G⁺ cells that were pre-treated with CPI-613 or DMSO. Results showed that inhibiting PDH with CPI-613 reversed the Lal^{-/-} Ly6G⁺ cells' suppressive activity on T cell proliferation versus Lal^{+/+} Ly6G⁺ cells (FIG. 3C). Next, Lal^{+/+} or Lal/Ly6G⁺ cells after CPI-613 pre-treatment were in vitro co-cultured with B16 melanoma or LLC cells or in vivo co-injected with B16 melanoma cells subcutaneously into Lal^{+/+} recipient mice. CPI-613 pre-treated Lal^{-/-} Ly6G⁺ cells showed reduced capabilities in promoting tumor cell proliferation when co-cultured with B16 melanoma and LLC cells in vitro (FIG. 3D) and stimulating tumor growth when co-injected with B16 melanoma cells in vivo (FIG. 3E). Since CPI-613 also blocks α-ketoglutarate dehydrogenase in the TCA cycle, a specific siRNA knockdown of PDH was performed, which was shown in FIG. 3F. Similar to the effects of CPI-613, knockdown of PDH in Lal^{-/-} Ly6G⁺ cells not only decreased their ROS production (FIG. 3G), but also reduced their ability in stimulating tumor cell proliferation (FIG. 3H). Interestingly, PD-L1 expression was up-regulated in bone marrow-derived Lal^{-/-} Ly6G⁺ cells by Western blot analysis versus Lal^{+/+} Ly6G⁺ cells (FIG. 3I). Similar to the metabolic enzyme genes involved in glycolysis and the citrate cycle, the PD-L1 gene (Cd274) were upregulated in cluster 123 and downregulated in cluster 0468 of Lal^{-/-} Ly6G⁺ cells versus Lal^{+/+} Ly6G⁺ cells. In vivo CPI-613 treatment effectively reduced PD-L1 expression by flow cytometry in Lal^{-/-} Ly6G⁺ cells versus Lal^{+/+} Ly6G⁺ cells (FIG. 3J).

### Example 4 Decrease of LAL Expression in Myeloid Subsets of NSCLC Patients

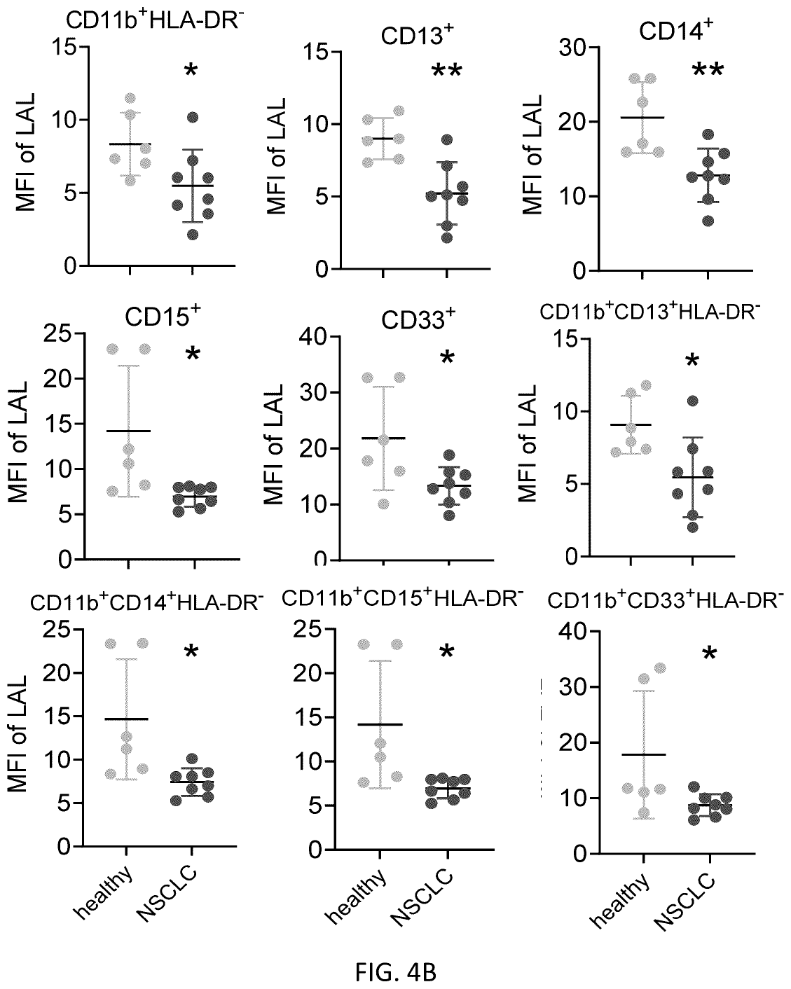

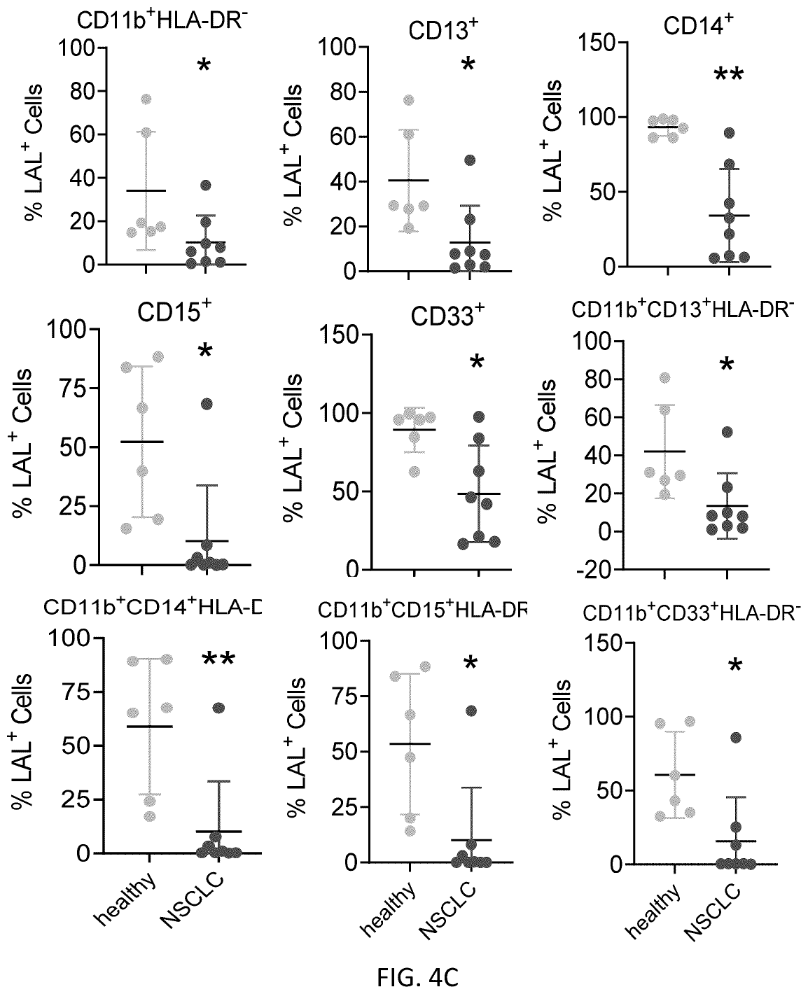

In the Lal^{-/-} mouse model, expansion of MDSCs is a pre-existing condition in favor of tumor growth. We recently reported downregulation of LAL gene expression in various human cancer forms by data mining analyses of The Cancer Genome Atlas (TCGA) database, and in the whole blood cells of non-small lung cancer (NSCLC) patients versus healthy subjects by flow cytometry. It is very inconvenient to use tumor-infiltrating MDSCs for the diagnosis/prognosis purposes without going through biopsy for lung cancer patients. In comparison, it is relatively easy to obtain blood samples from patients for diagnosis/prognosis. Here, we further assessed LAL expression in various blood myeloid subsets of NSCLC patients by flow cytometry. The LAL protein level was downregulated in blood CD13⁺, CD14⁺, CD15⁺, and CD33⁺ myeloid subsets from NSCLC patients versus healthy subjects (FIG. 4A; FIG. 4B). Further analyses demonstrated downregulation of the LAL protein level in CD11b⁺HLA-DR⁻ double-gated myeloid cells, and in CD11b⁺CD13⁺HLA-DR⁻, CD11b⁺CD14⁺HLA-DR⁻, CD11b⁺CD15⁺HLA-DR⁻ and CD11b⁺CD33⁺HLA-DR⁻ triple-gated myeloid subsets of NSCLC patients versus healthy subjects (FIG. 4A; FIG. 4B). The percentages of LAL⁺ cells were also reduced in the above myeloid subsets (FIG. 4C). Therefore, the levels of LAL protein expression in CD13⁺/CD14⁺/CD15⁺/CD33⁺ myeloid subsets can be used as potential negative biomarkers for diagnosis and prognosis of NSCLC.

### Example 5 Changes of Myeloid Subsets in Patients with NSCLC

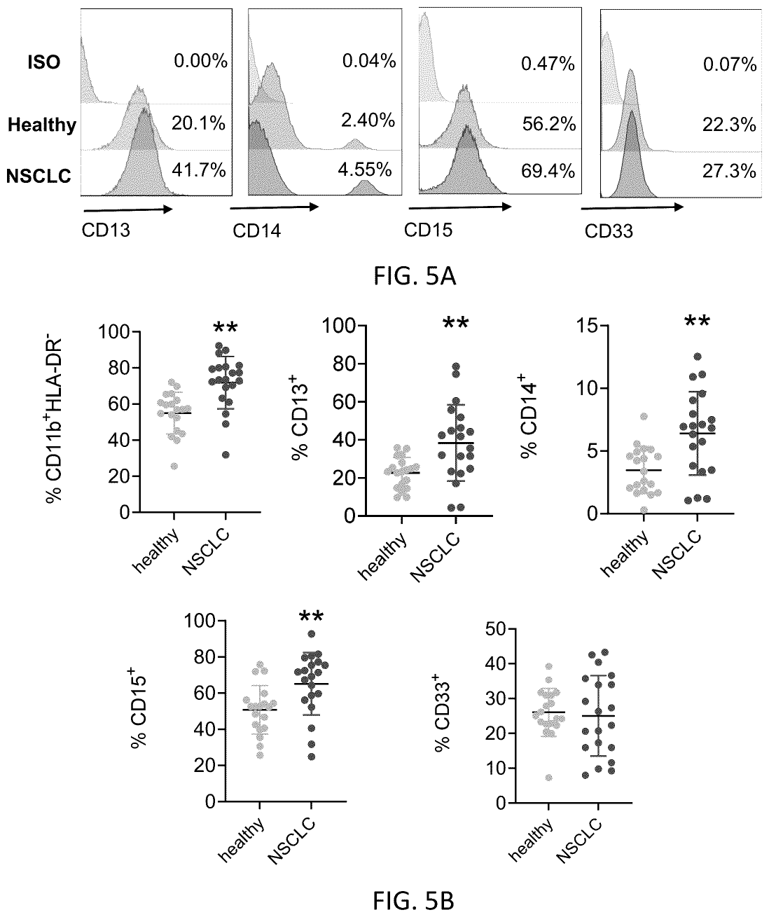

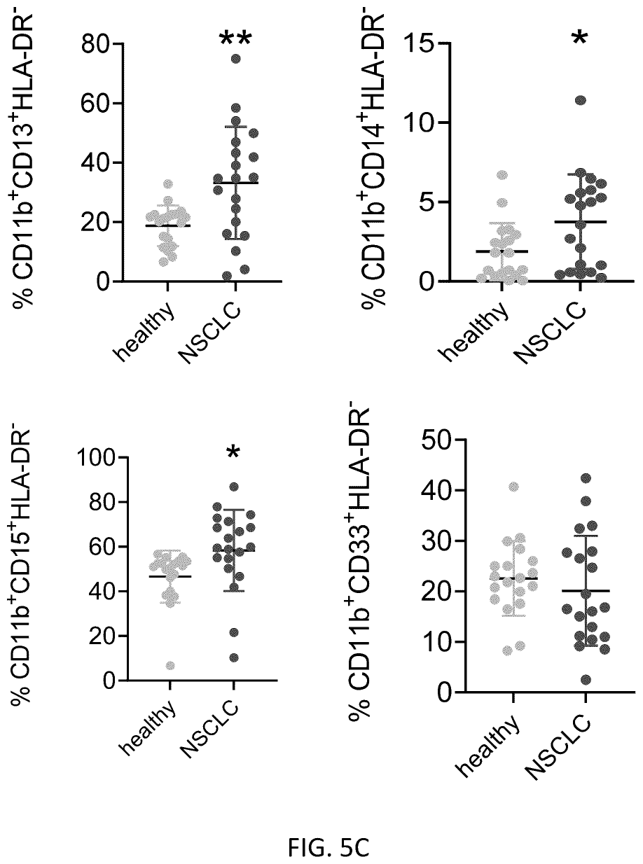

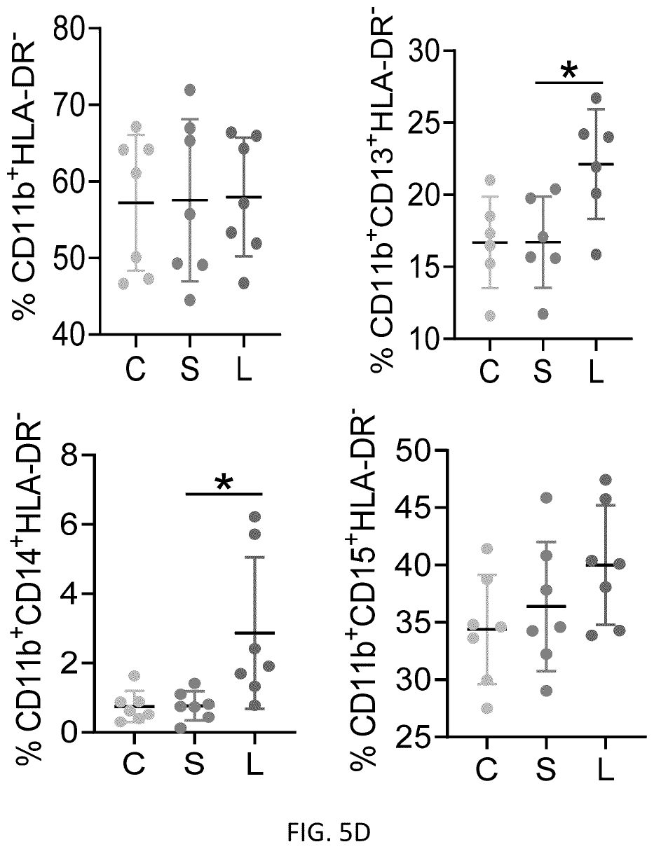

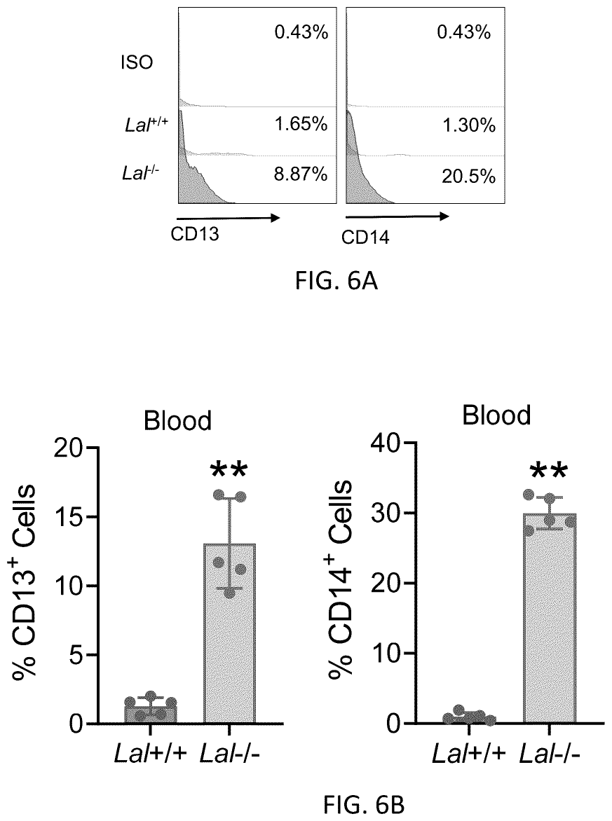

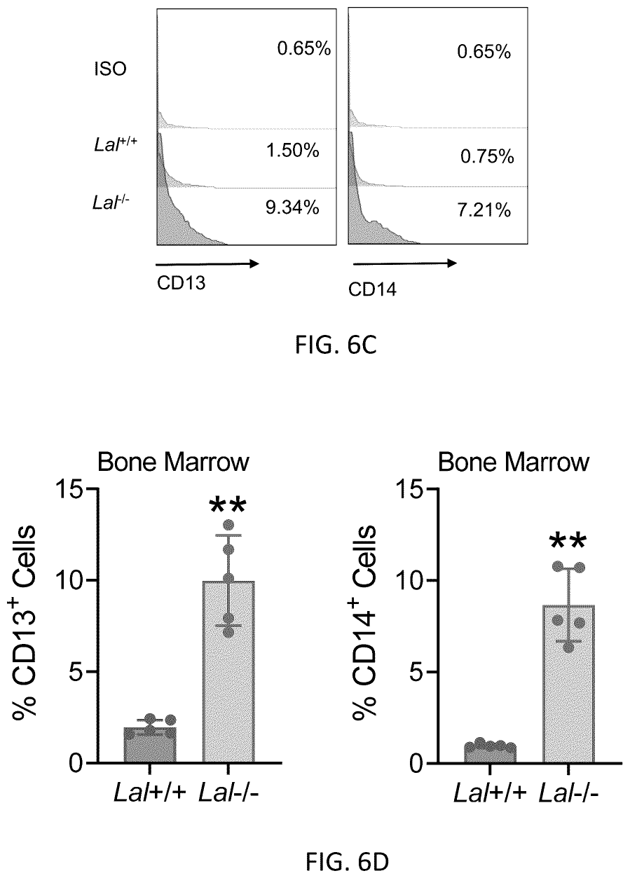

Not Only LAL serves as a biomarker, various myeloid subsets in the blood can also serve as potential indicators for diagnosis and prognosis of NSCLC. MDSCs in the human blood are a heterogeneous population (HLA-DR⁻/CD11b⁺, CD13⁺, CD14⁺, CD15⁺, CD33⁺, CD11c⁺ etc.). FIG. 5A and FIG. 5B showed that the percentages of CD11b⁺HLA-DR⁻, CD13⁺, CD14⁺, and CD15⁺ myeloid cells were all increased in NSCLC patients versus healthy subjects. The percentages of CD33⁺ myeloid cells did not change obviously in the leucocytes (FIG. 5A; FIG. 5B). Furthermore, in CD11b⁺HLA-DR⁻ double-gated myeloid cells, the percentages of CD11b⁺CD13⁺HLA-DR⁻, CD11b⁺CD14⁺HLA-DR⁻ and CD11b⁺CD15⁺HLA-DR⁻ MDSC subsets were all significantly increased in NSCLC patients, while there was no change in the percentages of CD11b⁺CD33⁺HLA-DR⁻ MDSC subset (FIGS. 5C, 5D). Therefore, CD13⁺/CD14⁺/CD15⁺ myeloid subsets serve as potential indicators for diagnosis and prognosis of NSCLC patients. The percentages of CD13⁺ and CD14⁺ cells were also increased in the blood and bone marrow of Lal^{-/-} mice (FIG. 6A; FIG. 6B; FIG. 6C; FIG. 6D). To definitively show whether LAL blockade induces differentiation of human myeloid cells, leucocytes from healthy subjects were treated with LAL specific inhibitor Lalistat2 for flow cytometry analysis. Results showed that blocking the LAL activity indeed increased the percentages of CD11b⁺CD13⁺HLA-DR⁻ and CD11b⁺CD14⁺HLA-DR⁻ MDSC subsets. The induction of CD11b⁺CD15⁺HLA-DR⁻ MDSC subset did not reach statistical significance (FIG. 5D).

### Example 6 Upregulation of Metabolic Enzymes in Patients with NSCLC

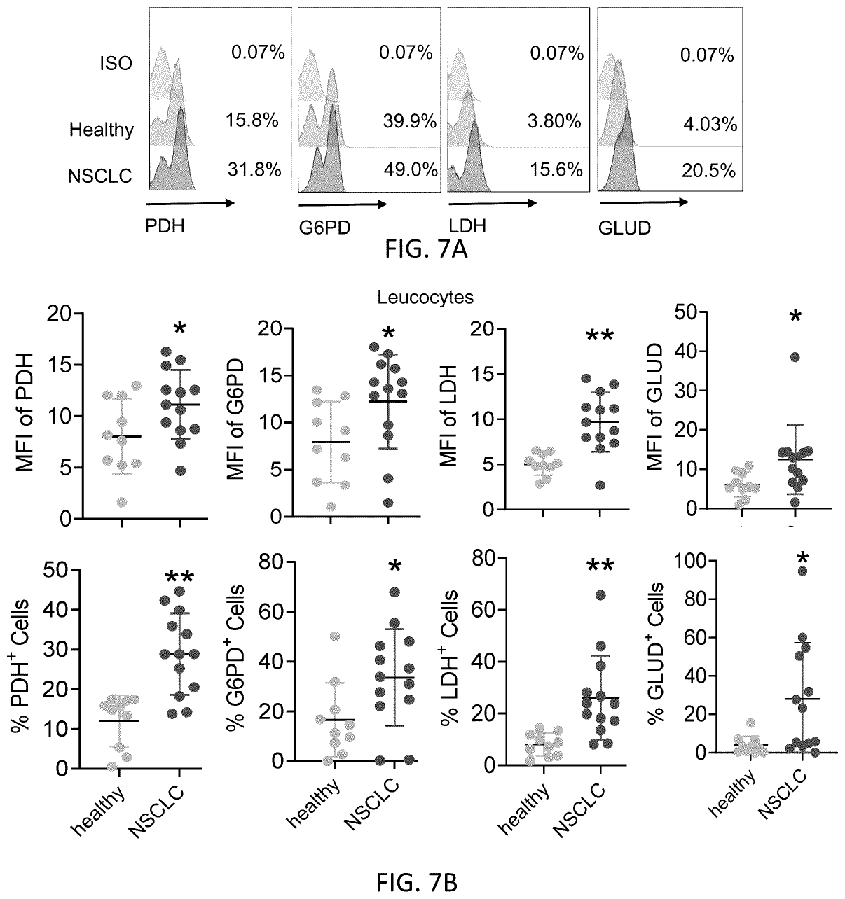

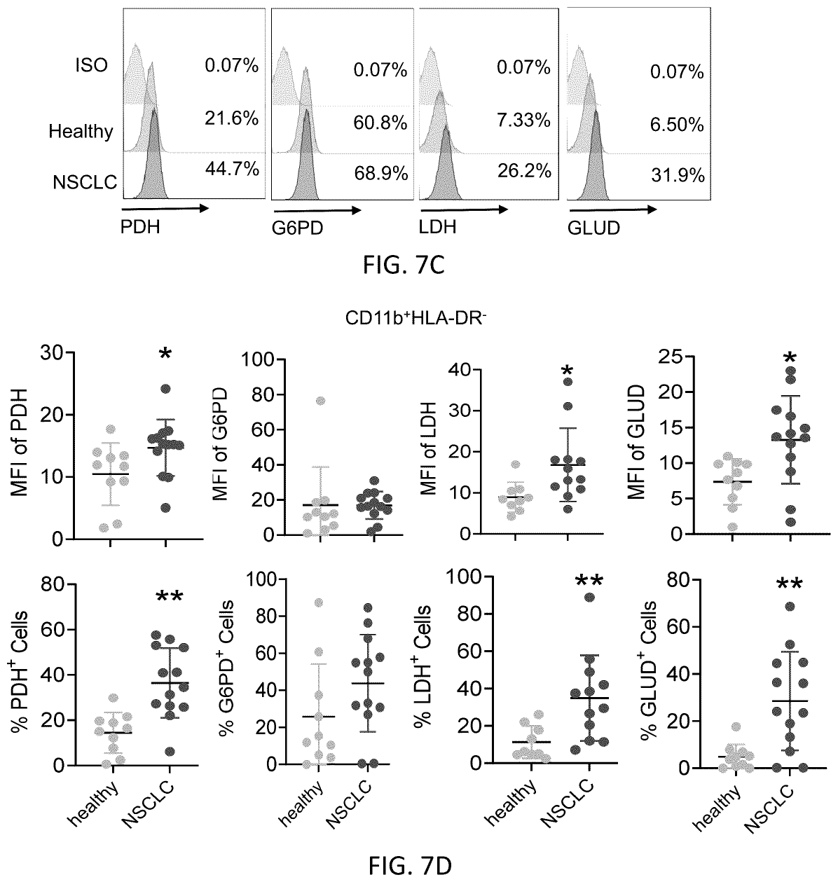

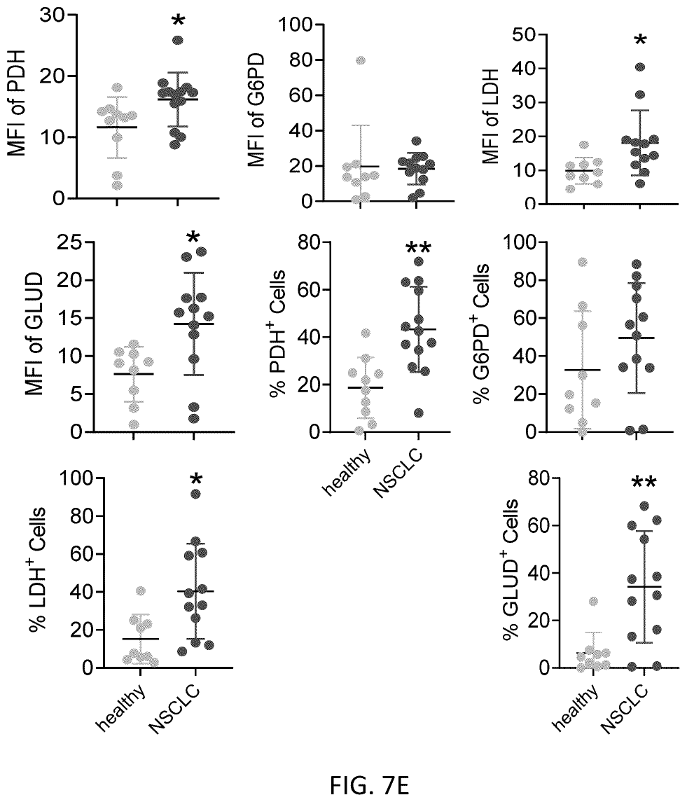

As demonstrated in mice, LAL deficiency causes MDSCs expansion through metabolic reprogramming as a mechanism. To expand this observation into human, increased mean fluorescent intensity (MFI) and percentage of glucose metabolic enzymes PDH, G6PD, and LDH were observed in leucocytes of NSCLC patients versus healthy subjects (FIG. 7A; FIG. 7B). Among amino acid pathways, GLUD was also increased in the leucocytes of NSCLC patients versus healthy subjects (FIG. 7A; FIG. 7B). Both MFI and percentages of PDH⁺, LDH⁺ and GLUD⁺ cells were upregulated in CD11b⁺HLA-DR⁻ and CD11b⁺CD13⁺HLA-DR⁻ myeloid subsets (FIG. 7C; FIG. 7D; FIG. 7E), except G6PD⁺ cells, confirming a metabolic shift towards glucose and amino acid utilization in myeloid subsets of NSCLC patients versus healthy subjects. Therefore, the metabolic enzymes serve as potential biomarkers for NSCLC diagnosis and prognosis.

### Example 7 Checkpoint Inhibitor Treatment in Patients with NSCLC

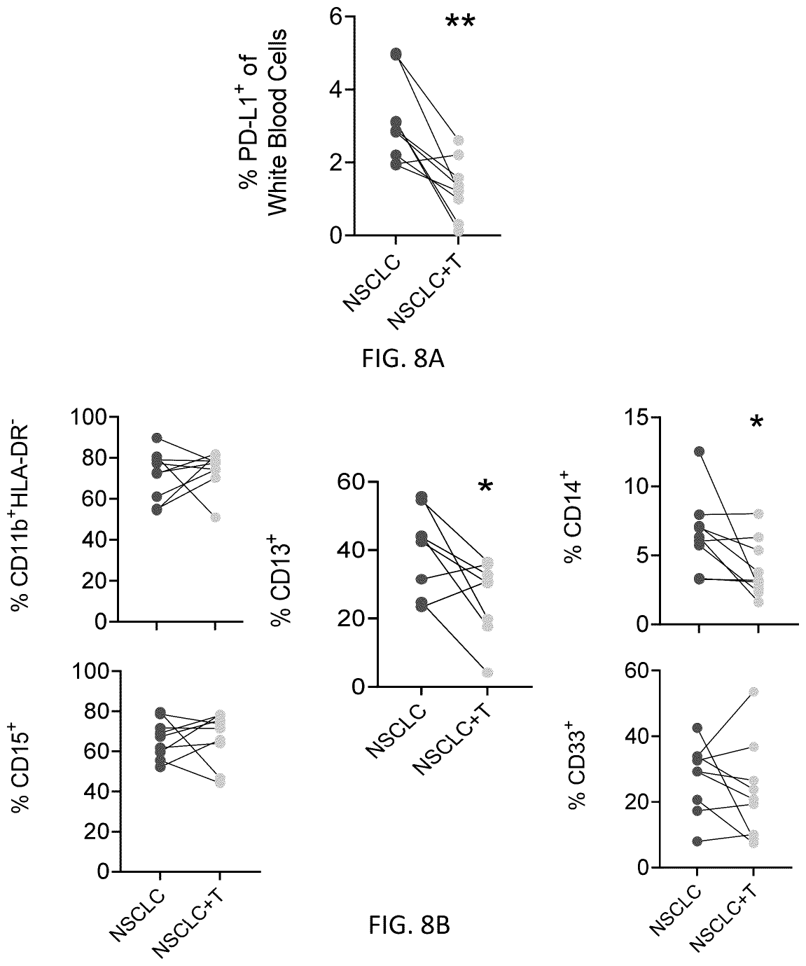

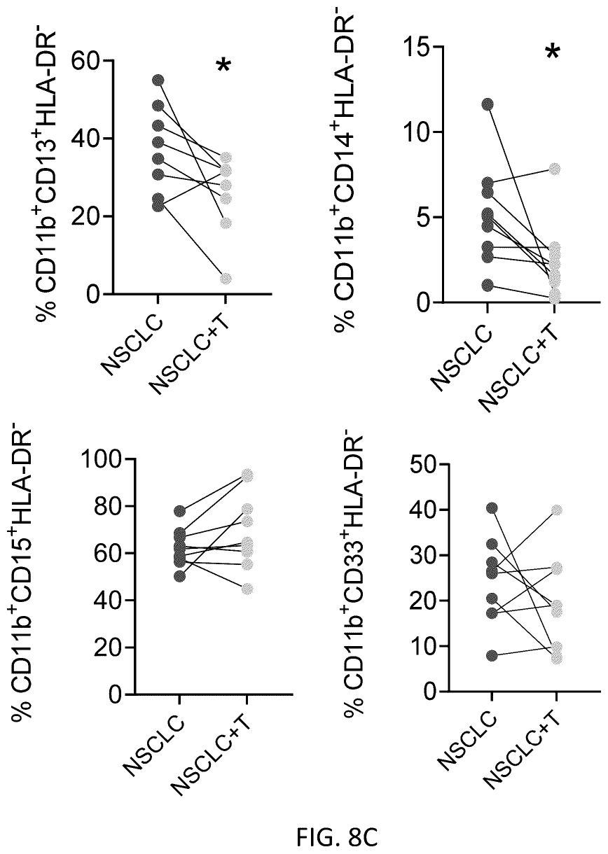

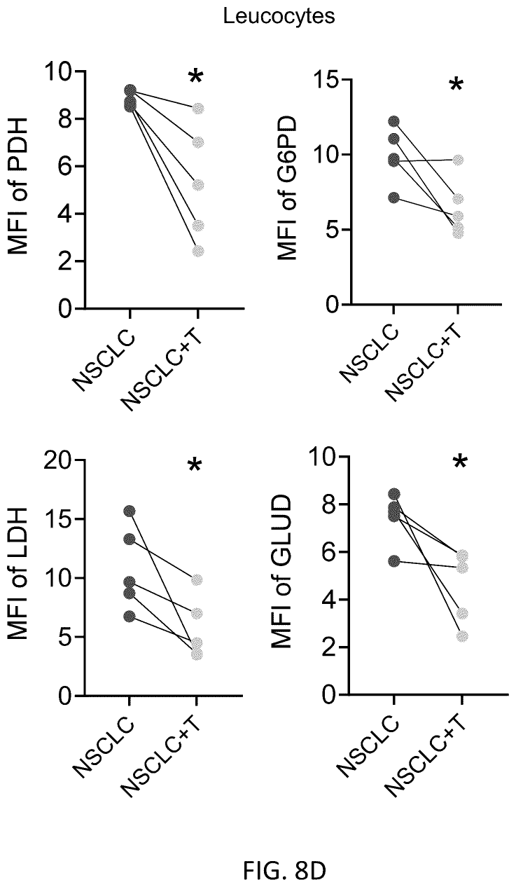

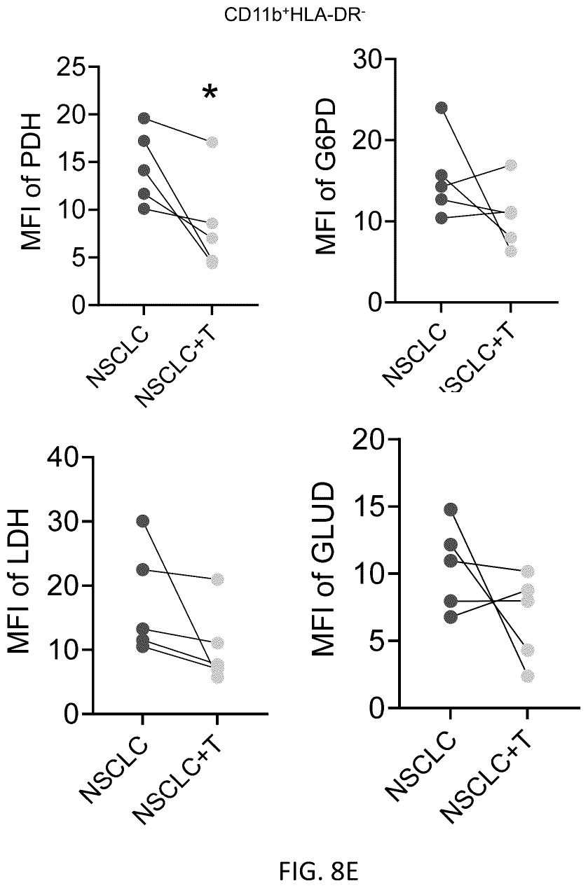

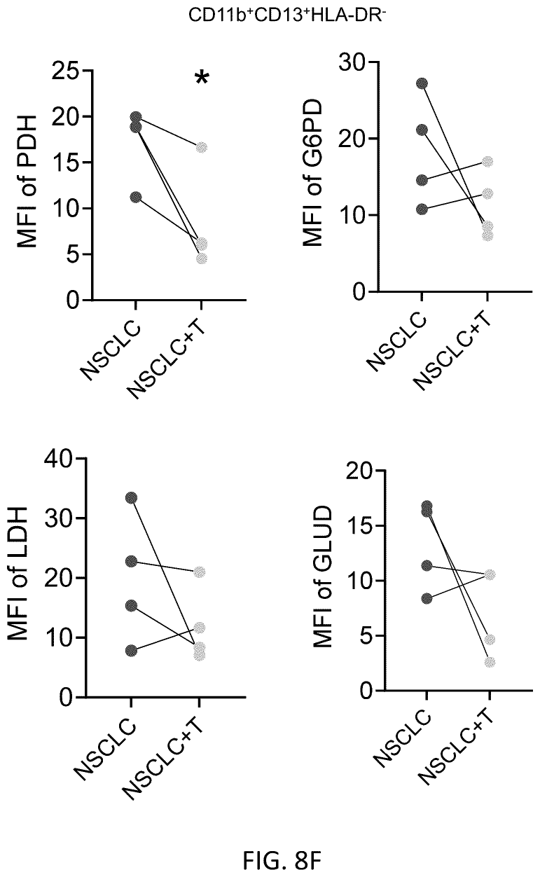

In Lal^{-/-} mice, increased expression of PD-L1 was observed in Ly6G⁺ cells that function as immune suppression and tumor stimulation. Antibody-based therapeutics targeting PD-1/PD-L1 have shown clinical benefits in multiple tumor types in human. To see which myeloid subsets respond to the treatment of PD-1 checkpoint blockade, the profiles of myeloid subsets were compared in NSCLC patients with PD-L1 Tumor Proportion Score≥10%. FIG. 8A showed that the PD-L1 level was decreased in the leucocytes of NSCLC patients after anti-PD-1 treatment (FIG. 9A). The percentages of CD13⁺ and CD14⁺ myeloid cells, but not CD15⁺, CD33⁺ or CD11b⁺HLA-DR⁻ cells, were reduced significantly after anti-PD-1 treatment (FIG. 8B). Moreover, in CD11b⁺HLA-DR⁻ double-gated myeloid subsets, anti-PD-1 treatment downregulated the percentages of CD11b⁺CD13⁺HLA-DR⁻ and CD11b⁺CD14⁺HLA-DR⁻ myeloid subsets (FIG. 8C). Therefore, CD13⁺ and CD14⁺ myeloid subsets responded to the anti-PD-1 treatment in NSCLC patients. When metabolic enzymes were examined in myeloid subsets, expression of PDH, G6PD, LDH and GLUD were all decreased in the leucocytes of NSCLC patients versus healthy subjects after treatment (FIG. 8D). With limited patients available, the CD13⁺ myeloid subset was chosen for further analysis. In CD11b⁺HLA-DR⁻ and CD11b⁺CD13⁺HLA-DR⁻ myeloid subsets, MFI of PDH expression was downregulated while others were inconclusive after anti-PD-1 treatment (FIG. 8E; FIG. 8F). PDH in blood myeloid cells can be used as a potential biomarker for diagnosis and prognosis of NSCLC. The clinical outcomes of this trial cohort showed 7 patients with partial responses (tumor shrunk for some period), 2 patients with stable condition (tumor neither grew nor shrunk), while 1 patient with progression after 2 cycles of anti-PD-1 treatment.

The contents of the above-named references are incorporated by reference in their entireties herein.

1. A method of treating a cancer in a subject comprising: i) inhibiting a tumor microenvironment comprising: a) administering a therapeutically effective amount of a composition including a lysosomal acid lipase (LAL) replacement therapy, a pyruvate dehydrogenase (PDH) inhibitor, a programmed death-ligand 1 (PD-L1) inhibitor, or a combination thereof; and ii) administering an anti-tumor treatment.

2. The method of claim 1, wherein the tumor microenvironment includes myeloid derived suppressor cells (MDSCs).

3. The method of claim 2, wherein the MDSCs are characterized by the expression of CD11b⁺, CD13⁺, CD14⁺, CD15⁺, CD33⁺, HLA-DR⁻, or a combination thereof.

4. The method of claim 2, wherein the MDSCs are characterized by a reduced expression level of lysosomal acid lipase (LAL) compared to a myeloid cell not in a tumor microenvironment.

5. The method of claim 1, wherein the composition is formulated for administration as a liquid or a powder.

6. The method of claim 5, wherein the composition is a pharmaceutical composition or salt thereof.

7. The method of claim 1, wherein the composition includes a lysosomal acid lipase (LAL) replacement therapy.

8. The method of claim 7, wherein the LAL replacement therapy comprises Sebelipase alfa.

9. The method of claim 1, wherein the composition includes a PDH inhibitor.

10. The method of claim 1, wherein the composition includes a PD-L1 inhibitor.

11. The method of claim 1, wherein the composition includes an LAL replacement therapy and a PDH inhibitor.

12. The method of claim 1, wherein the composition includes an LAL replacement therapy and a PD-L1 inhibitor.

13. The method of claim 1, wherein the composition is administered intravenously, orally, topically, or through inhalation.

14. The method of claim 1, wherein the anti-tumor treatment is selected from surgery, chemotherapy, radiotherapy, immunotherapy, or a combination thereof.

15. The method of claim 1, wherein the composition comprises an LAL replacement therapy and the anti-tumor treatment is an immunotherapy.

16. The method of claim 1, wherein the composition is administered before the anti-tumor treatment.

17. The method of claim 1, wherein the composition is administered after the anti-tumor treatment.

18. The method of claim 1, wherein the composition comprises an LAL replacement therapy and a PD-L1 inhibitor.

19. The method of claim 1, wherein the composition comprises an LAL replacement therapy and a PDH inhibitor.

20. The method of claim 1, wherein the cancer is lung cancer.
